(19) United States
(12) Patent Application Publication
(10) Pub. No.: US 2003/0082551 A1
(43) Pub. Date: May 1, 2003

(54) High-throughput gene cloning and phenotypic screening

(76) Inventors: David A. Zarling, Menlo Park, CA (US); Ron Caspi, Mountain View, CA (US); Kathryn M. Stephens, Fremont, CA (US); Roy G. Sergeant, San Lorenzo, CA (US); Christopher Lehman, Sunnyvale, CA (US); Sushma Pati, Woodside, CA (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/967,323

(22) Filed: Sep. 28, 2001

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

The invention relates to the use of high-throughput methods for gene targeting, recombination, phenotype screening and biovalidation of drug targets utilizing enhanced homologous recombination (EHR) techniques. These methods utilize robotically driven multichannel pipetters to perform liquid, particle, cell and organism handling, robotically controlled plate and sample handling platforms, magnetic probes and affinity probes to selectively capture nucleic acid hybrids, and thermally regulated plates or blocks for temperature controlled reactions.

### FIELD OF THE INVENTION

The invention relates to the use of high-throughput methods for gene targeting, recombination, phenotype screening and biovalidation of drug targets utilizing enhanced homologous recombination (EHR) techniques. These methods utilize robotically driven single or multichannel pipetters to perform liquid, particle, cell and organism handling, robotically controlled plate and sample handling platforms, and thermally regulated plates or blocks for temperature controlled reactions.

### BACKGROUND OF THE INVENTION

The Genome Project has produced thousands of expressed sequence tags (EST), however, the bottleneck in functional Genomics is the isolation of full-length gene clones and the determination of gene function. Functional Genomics covers the study of the action and interaction of gene products and their targets, thereby providing clues to reveal the relationship between patterns of gene expression and its pathological or other phenotypical consequence in cells, tissues and organisms. However, conventional approaches to gene and phenotypic screening for biovalidation of drug targets are hampered by low throughput processes that are inherently slow and labor intensive. The limitations are encountered at every step of the process from gene cloning, target identification, phenotypic screening and small molecule bioassays to drug and phenotypic biovalidation in cells and animals.

Homologous recombination (HR) is defined as the exchange of homologous or similar DNA sequences between two DNA molecules. An essential feature of HR is that the enzymes responsible for the recombination event can pair any homologous sequences as substrates. The ability of HR to transfer genetic information between DNA molecules makes targeted homologous recombination a very powerful method in genetic engineering and gene manipulation. HR can be used to add subtle mutations at known sites, replace wild type genes or gene segments or introduce completely foreign genes into cells. However, HR efficiency is very low in living cells and is dependent on several parameters, including the method of DNA delivery, how it is packaged, its size and conformation, DNA length and position of sequences homologous to the target, and the efficiency of hybridization and recombination at chromosomal sites. These variables severely limit the use of conventional HR approaches for gene evolution in cell-based systems. (Kucherlapati et al., 1984 PNAS USA 81:3153-3157; Smithies et al. 1985 Nature 317:230-234; Song et al. 1987 PNAS USA 84:6820-6824; Doetschman et al. 1987 Nature 330:576-578; Kim and Smithies 1988. Nuc. Acids. Res. 16:8887-8903; Koller and Smithies 1989. PNAS USA 86:8932-8935; Shesely et al. 1991 PNAS USA 88:4294-4298; Kim et al. 1991 Gene 103:227-233).

The frequency of HR is significantly enhanced by the presence of recombinase activities in cellular and cell free systems. Several proteins or purified extracts that promote HR (i.e., recombinase activity) have been identified in prokaryotes and eukaryotes (Cox and Lehman, 1987. Annu. Rev. Biochem. 56:229-262; Radding. 1982. Annual Review of Genetics 16:405-547; McCarthy et al. 1988. PNAS USA 85:5854-5858). These recombinases promote one or more steps in the formation of homologously-paired intermediates, strand-exchange, and/or other steps. The most studied recombinase to date is the RecA recombinase of Escherichia coli, which is involved in homology search and strand exchange reactions (Cox and Lehman, 1987, supra).

The E. coli RecA protein (Mr 37,842) catalyses homologous pairing and strand exchange between two homologous DNA molecules (Kowalczykowski et al. 1994. Microbiol. Rev. 58:401-465; West. 1992. Annu. Rev. Biochem. 61:603-640); Roca and Cox. 1990. CRC Cit. Rev. Biochem. Mol. Biol. 25:415-455; Radding. 1989. Biochim. Biophys. Acta. 1008:131-145; Smith. 1989. Cell 58:807-809). RecA protein binds cooperatively to any given sequence of single-stranded DNA with a stoichiometry of one RecA protein monomer for every three to four nucleotides in DNA (Cox and Lehman, 1987, supra). This forms unique right handed helical nucleoprotein filaments in which the DNA is extended by 1.5 times its usual length (Yu and Egelman 1992. J. Mol. Biol. 227:334-346). The phosphate backbone of DNA inside the RecA nucleoprotein filaments is protected against digestion by phosphodiesterases and nucleases. These nucleoprotein filaments, which are referred to as targeting polynucleotides, are crucial “homology search engines” which catalyze DNA pairing. Once the filament finds its homologous double-stranded target gene sequence, the DNA targeting polynucleotide strand invades the target and forms a hybrid DNA structure, referred to as a joint molecule or D-loop (DNA displacement loop) (McEntee et al. 1979. PNAS USA 76:2615-2619; Shibata et al. 1979. PNAS USA 76:1638-1642).

RecA protein is the prototype of a universal class of recombinase enzymes that promote DNA pairing reactions. Recently, genes homologous to E.coli recA (encoding the Rad51 family of proteins) were isolated from all groups of eukaryotes, including yeast and humans. The Rad51 protein promotes homologous pairing and strand invasion and exchange between homologous DNA molecules in a similar manner to the RecA protein (Sung. 1994. Science 265:1241-1243; Sung and Robberson. 1995. Cell 82:453-461; Gupta et al. 1997. PNAS USA 94:463-468; Baumann et al. 1996. Cell 87:757-766).

Enhanced homologous recombination (EHR) technology (utilizing nucleoprotein filaments) increases the efficiency and specificity of homologous DNA targeting and recombination in living cells and targeting to native double-stranded DNA in solution and in situ by utilizing complexes of DNA, recombinase protein, and DNA targets. These EHR gene targeting reactions proceed via multi-stranded DNA hybrid intermediates formed between the nucleoprotein filaments (as complementary single-stranded DNA or cssDNA targeting polynucleotides) and homologous gene targets. These kinetically-trapped multi-stranded hybrid DNA intermediates have been very well-characterized, are biologically active in enhancing homologous recombination and can tolerate significant heterologies, thus enabling the insertion of transgenes and the modification of host genes at virtually any selected site.

EHR methods and compositions have been used to target and alter substitutions, insertions and deletions in target sequences and are described; see U.S. application Ser. Nos. 08/381634; 08/882756, 09/301153; 08/781329; 09/288586; 09/209676; 09/007020; 09/179916; 09/182102; 09/182097; 09/181027; 09/260624; 09/373,347; 09/306,749; No. 60/153,795; and international application nos. US97/19324; US98/26498; US98/01825, all of which are expressly incorporated by reference in their entirety.

Accordingly, it is an object of the invention to provide high-throughput methods for gene targeting, recombination, phenotype screening and biovalidation of drug targets utilizing EHR techniques. These methods utilize robotically driven multichannel pipetters to perform liquid, particle, cell and organism handling, robotically controlled plate and sample handling platforms, and thermally regulated plates or blocks for temperature controlled reactions.

### SUMMARY OF THE INVENTION

In accordance with the objects outlined herein, the present invention provides methods of cloning a target nucleic acid using an enhanced homologous recombination (EHR) composition comprising a recombinase, a first and a second targeting polynucleotides, and a separation moiety. The first polynucleotide comprises a fragment of the target nucleic acid and is substantially complementary to the second target polynucleotide. The EHR composition is contacted with a nucleic acid library or other composition of nucleic acid, under conditions wherein said targeting polynucleotides can hybridize to the target nucleic acid. The target nucleic acid is isolated; and at least one of these steps utilizes a robotic system.

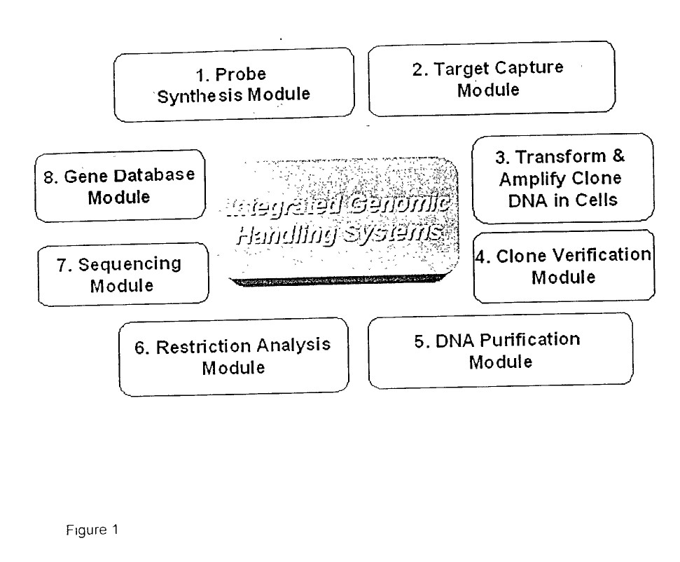

Such a robotic system can include, but is not limited to, the following components (FIG. 1):

1. A targeting polynucleotide synthesis module

2. A target capture module

3. A transformation and amplification module

4. A clone verification module

5. A DNA purification module

6. A restriction analysis module

7. A DNA sequencing module

8. A computer database module

In an additional aspect, the methods further comprise making a library of nucleic acid variants of the target nucleic acid. These variants are then introduced into a target library and phenotypically screened.

In a further aspect, the methods further comprise making a plurality of cells comprising a mutant target nucleic acid and adding a library of candidate agents to the cells. The effect of the candidate agents on the cells is then determined, with optionally determining the effect of the candidate agent on the gene products of the nucleic acids.

In an additional aspect, the methods of the invention utilize many robotic systems comprising of computer workstations programmed to manipulate devices selected from the group consisting of thermal cyclers, 8-, 96-, or 384-tip multichannel liquid handlers, sample handlers, plate piercers, plate handlers, robotic arms, gel loading systems, barcode readers and applicators, temperature controlled plate stations, automated transformation systems, gene sequencers, colony pickers, magnetic bead processing stations, plate fillers, plate washers, plate shakers, vacuum filtration systems, cell sorters, incubators, light microscopes, fluorescence microscopes, microplate spectrofluorimeters, microplate spectrophotometers, microplate luminometers, CCD cameras and combinations thereof.

In a further aspect, the invention provides methods of high throughput integrated Genomics comprising a plurality of enhanced homologous recombination (EHR) compositions as outlined herein. The EHR compositions are contacted with one or more nucleic acid sample(s) under conditions wherein the targeting polynucleotides hybridize to one or more target nucleic acid member(s) of one or more libraries or other compositions. The target nucleic acid(s) are then isolated. The isolated target nucleic acids may comprise a gene identical to the targeting polynucleotide, as well as single-nucleotide polymorphisms, a gene family, or a haplotype.

In an additional aspect, the invention provides methods comprising identifying a cell(s), embryo(s), organism(s) having an altered phenotype induced by a biological activity of the expressed target nucleic acid, wherein the identifying is done using a robotic system. The expressed target sequence may be sequenced and/or mapped.

In a further aspect, the invention provides robotic systems comprising means for producing a plurality of enhanced homologous recombination compositions; means for contacting the compositions with a cellular library or other composition of nucleic acid under conditions wherein the enhanced homologous recombination compositions hybridize to one or more target nucleic acid members of the nucleic acid composition; means for isolating said target nucleic acid(s); means for producing a library of mutant target nucleic acid(s); means for nucleotide sequencing said target nucleic acid(s); means for determining the haplotype of said target nucleic acid; means for introducing said target nucleic acid(s) into host cells; means for expressing said target nucleic acid(s) in said cells; means for identifying one or more cell(s) having an altered phenotype induced by a biological activity of said expressed target nucleic acid(s); means for contacting said cell(s) with a library of candidate bioactive agents; and means for identifying one or more bioactive agent(s) that modulate a biological activity of said expressed target nucleic acid(s).

### DETAILED DESCRIPTION OF THE DRAWINGS

FIG. 1. A Genomic handling system capable of automating the process of EHR-based gene cloning is composed of eight modules.

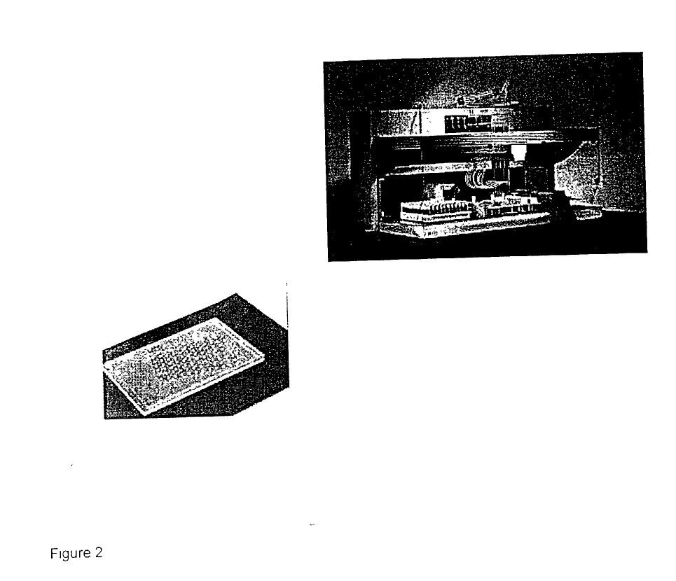

FIG. 2. An example of hardware for module 1: Targeting polynucleotide synthesis module. The example includes a Tecan Genesis liquid handler equipped with a RoMa robotic arm and an integrated thermal cycler block (alpha block of a MJ Research DNA Engine thermal cycler). The deck is equipped with a magnetic bead-processing unit (MagBead unit made by Tecan), A custom plate piercer made by Tecan, and a Tecan Plate sealer. It also has a custom gel loading device designed for MADGE (Multiple Array Diagonal Gel Electrophoresis) prepared by MadgeBio, UK.

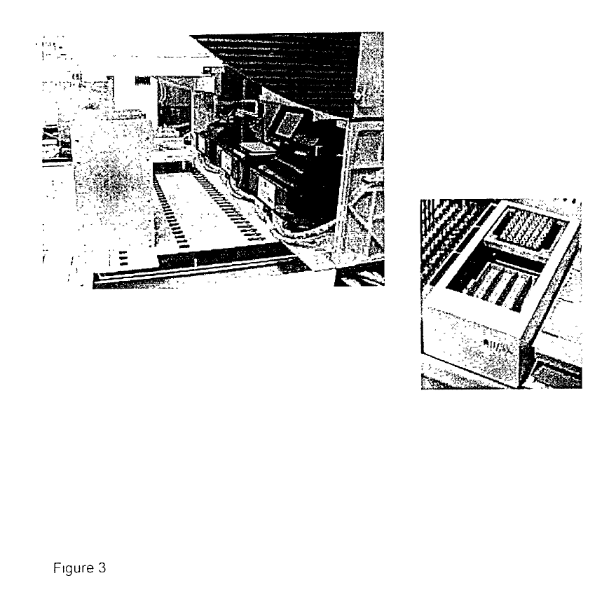

FIG. 3. An example of hardware for module 2: Target capture. The example includes a Tecan Genesis liquid handler equipped with a RoMa robotic arm and integrated thermal cycler blocks (alpha blocks of a MJ Research DNA Engine thermal cycler). The deck is equipped with a magnetic bead-processing unit (MagBead unit made by Tecan), a custom plate piercer made by Tecan, and a Tecan Plate sealer. It also includes a shaker.

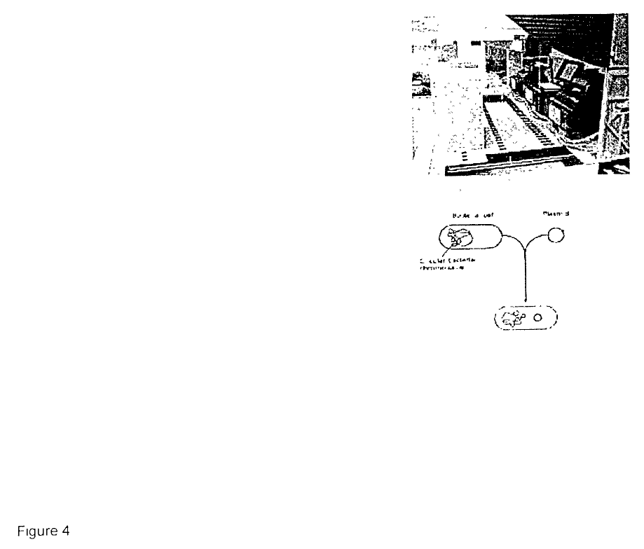

FIG. 4. An example of hardware for module 3: Transformation and amplification module. The example includes a Tecan Genesis liquid handler equipped with a RoMa robotic arm and integrated thermal cycler block (alpha block of a MJ Research DNA Engine thermal cycler). The deck is also equipped with a chilled position.

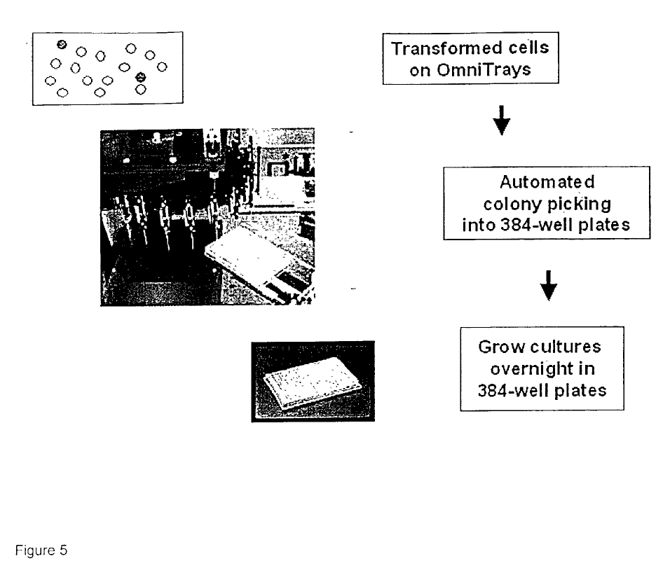

FIG. 5. Colonies on agar plates are picked by a colony picker (in this example, a GeneMachines Mantis) into 384-well culture plates.

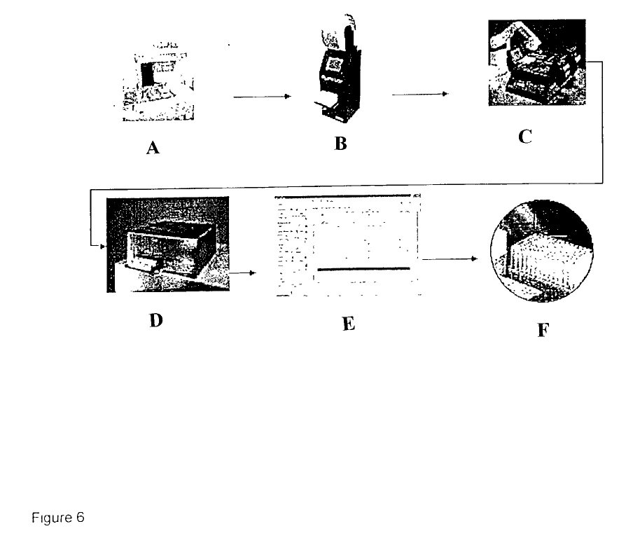

FIG. 6. An example of hardware for module 4: Clone verification. This example includes a Tecan Genesis liquid handler equipped with a RoMa robotic arm and a Tecan Genmate equipped with a 384-channel pipettor head. An Orca robotic arm on a track (Beckman Coulter) integrates the liquid handlers with a Velocity 11 plate sealer, a plate piercer, a number of MJ Research DNA Tetrad thermal cyclers, and a Tecan SpectraFluor Plus plate reader.

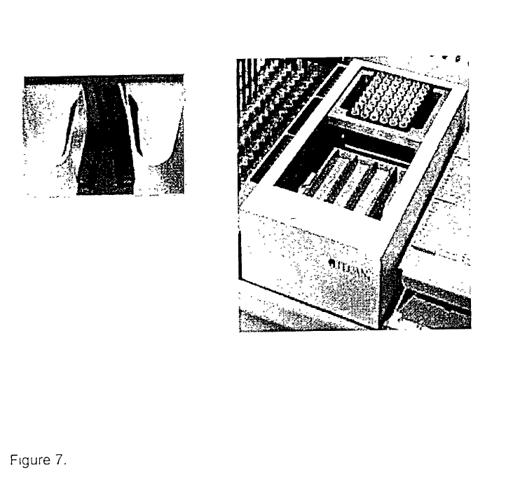

FIG. 7. An example of hardware for module 5: DNA purification. The example includes a Tecan Genesis liquid handler equipped with a RoMa robotic arm, a magnetic bead-processing unit (MagBead unit made by Tecan) and a shaker. A centrifuge for centrifuging microtiter plates is also necessary.

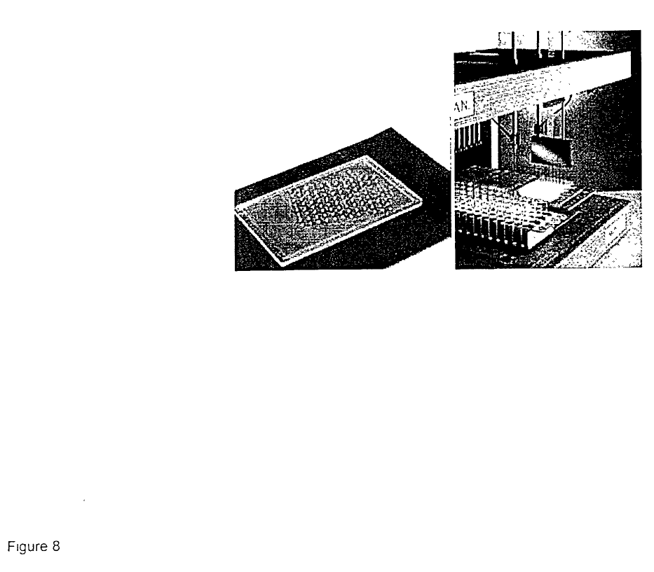

FIG. 8. An example of hardware for module 6: Restriction analysis. The example includes a Tecan Genesis liquid handler equipped with a RoMa robotic arm and integrated thermal cycler blocks (alpha blocks of a MJ Research DNA Engine thermal cycler). The deck is equipped with a chilled position and a custom gel-loading device.

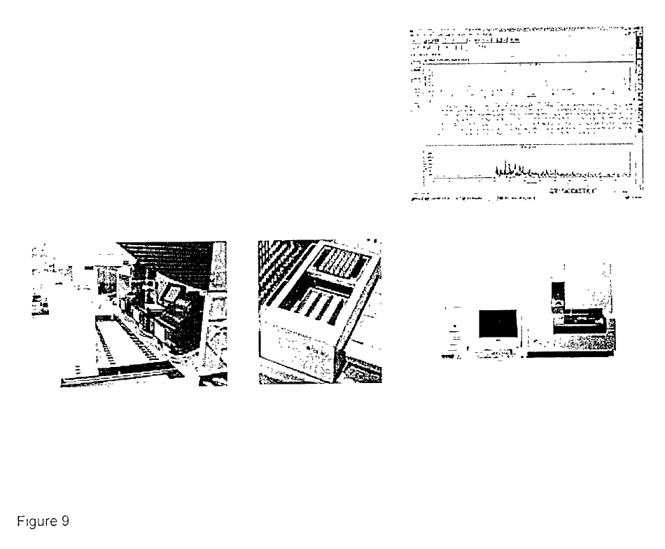

FIG. 9. An example of hardware for module 7: DNA Sequencing. The example includes a Tecan Genesis liquid handler equipped with a RoMa robotic arm and integrated thermal cycler blocks (alpha blocks of a MJ Research DNA Engine thermal cycler). The deck is equipped with a shaker, a magnetic bead-processing unit (MagBead unit made by Tecan), a custom plate piercer made by Tecan, and a Tecan Plate sealer.

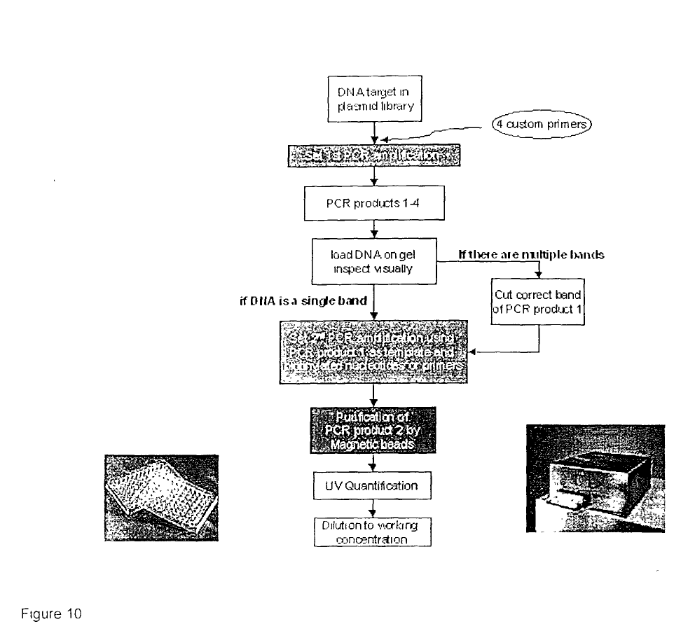

FIG. 10. A flow-chart depicting the process of automated library validation and targeting polynucleotide synthesis

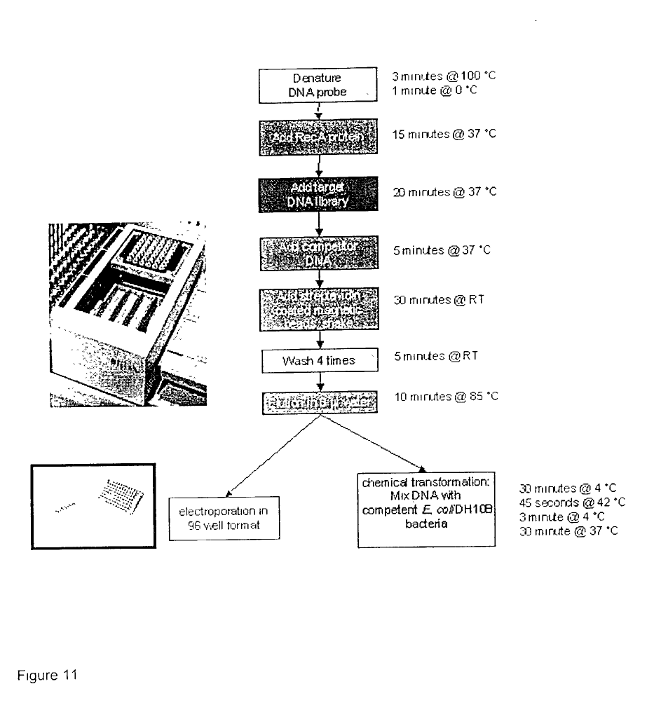

FIG. 11. A flow-chart depicting the process of automated target capture cell transformation.

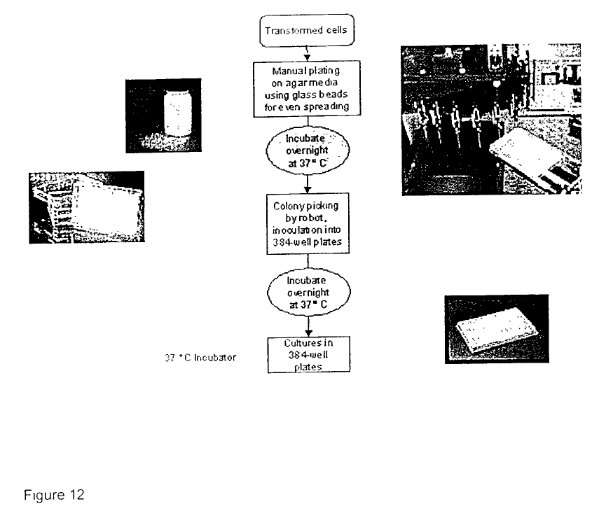

FIG. 12. A flow-chart depicting the process of automated colony picking into 384-well culture plates.

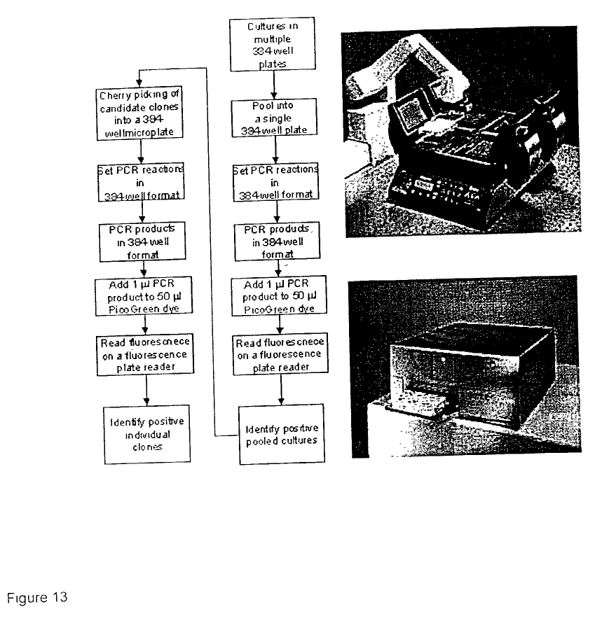

FIG. 13. A flow-chart depicting the process of automated clone verification using PCR followed by PicoGreen assays. This process assumes pooling of cultures is required.

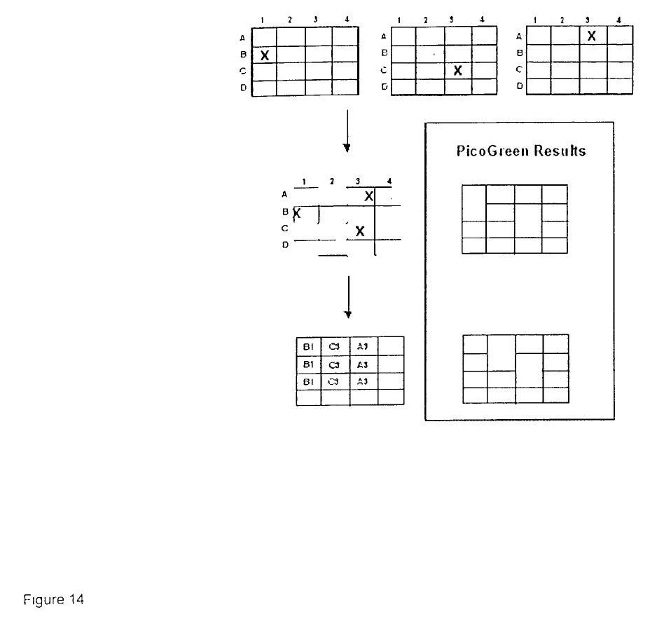

FIG. 14. An explanation of the pooling process which is used to speed screening of multiple culture plates containing clones harboring the same target.

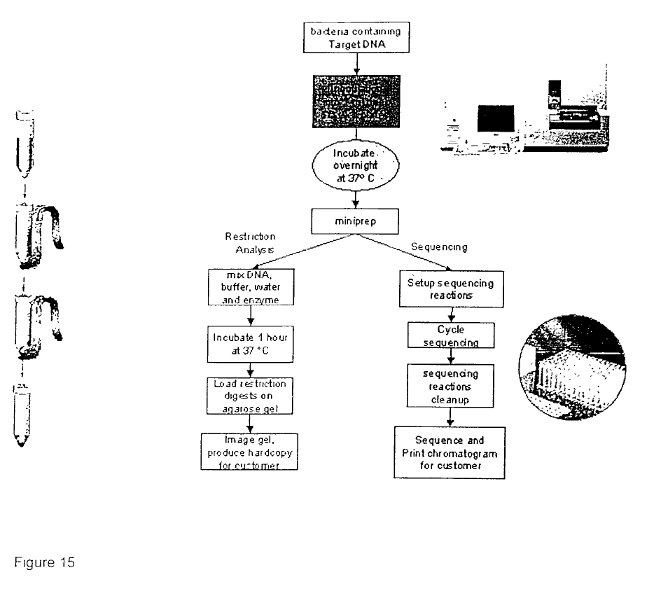

FIG. 15. A flow-chart depicting the process of automated plasmid purification, restriction analysis and sequencing.

### DETAILED DESCRIPTION

The present invention is directed to the use of enhanced homologous recombination (EHR) techniques in combination with high-throughput microprocessor controlled robotic systems. The EHR technology enables the rapid generation of recombinants and alleviates the rate limiting bottlenecks in target-driven drug discovery. The recombinase-nucleic acid targeting polynucleotides are designed to specifically bind to the target DNA sequence(s) and replace, insert or delete the designated nucleotide(s) within the gene or highly-relevant gene families. See U.S. application Ser. Nos. 08/381634; 08/882756; 09/301153; 08/781329; 09/288586; 09/209676; 09/007020; 09/179916; 09/182102; 09/182097; 09/181027; 09/260624; 09/373,347; 09/306,749; No. 60/153,795; and international application nos. US97/19324; US98/26498; US98/01825, all of which are expressly incorporated by reference in their entirety.

Previous work emphasized that the stringency of the recombinase-mediated homologous DNA targeting can be reduced by using nucleoprotein filaments formulated with degenerate targeting polynucleotides. The average sequence derived from related sequences is called the consensus sequence, as further outlined below. Since Enhanced Homologous Recombination (EHR) can tolerate up to 30% mismatches between the between single-stranded DNA (ssDNA) targeting polynucleotides and double-stranded DNA (dsDNA) molecules, cDNA targeting polynucleotides that are directed to these consensus sequences can simultaneously target many members of a related gene family. The isolation of novel related genes by EHR cloning can be performed by using a single ssDNA targeting polynucleotide species with a consensus sequence to a functional domain (homology motif tag (HMT)), by using targeting polynucleotides with limited homology, or by using targeting polynucleotides with degenerate consensus sequences. In addition, gene targeting with specific heterologies within the cssDNA targeting polynucleotides allows for rapid gene targeting and cloning, generation of gene family specific libraries, and evolution of gene family members. Sequence analysis of the isolated cDNAs and genomic DNA allows diagnostic testing for single and multiple nucleotide polymorphisms, loss of heterozygosity (LOH), and other chromosomal abnormalities.

EHR can be used to repair mutant genes, alter genes, or interrupt normal gene function to identify critical genes, gene products and pathways active in the cells and organisms by analyzing phenotypic changes and altered protein states and interactions. The gene and protein expression patterns, correlations and delayed correlations in model systems can be used to identify and verify the function and importance of key elements in a disease process. EHR is a powerful technique that can be used to repair genetic defects that cause or contribute to disease. EHR can be developed for use in diseases including hemophilia, cardiovascular disease, muscular dystrophy, cystic fibrosis and other genetically based diseases. This technique is technically feasible and applicable within plant, animal, human, and bacterial cells.

EHR has significant advantages over the conventional methods of random mutagenesis to generate genetic variants. The advantages of recombinase mediated gene cloning and phenotyping are 1.) Increased efficiency of recombinant formation to allow the generation of a vast number of genetic variants; 2.) Increased specificity of DNA targeting and recombination at the desired sites within the clone or gene in vitro, in living cells, and in situ, by utilizing complexes of ssDNA, recombinase protein, and dsDNA targets for homologous, non-random reactions; 3.) Simultaneous targeting, cloning, and phenotyping of multiple gene family members; because the recombinases can tolerate up to 30% mismatches between the ssDNA targeting polynucleotides and the dsDNA molecules, degenerate targeting polynucleotides can be used, and the stringency of targeting can be reduced; 4.) Multiple iterations of a modification/mutation can be tested.

EHR has been successfully used to modify genes in cells and animals, including bacteria, plants, zebra fish, mice and goats. These EHR gene-targeting reactions proceed via multi-stranded DNA hybrid intermediates formed between the nucleoprotein filaments (as complementary single-stranded DNA [cssDNA] targeting polynucleotides) and homologous gene targets. These kinetically-trapped multi-stranded hybrid DNA intermediates are very well-characterized, biologically active in enhancing homologous recombination and can tolerate significant heterologies, thus enabling the insertion of transgenes and the modification of host genes at virtually any selected site. Since cssDNA targeting polynucleotides are generally 200-500 bp long, this method is useful for generating cssDNA targeting polynucleotides starting from expressed sequence tags (ESTs), isolated exons or homologous sequence information.

In addition, RecA-mediated cloning has been done (Teintze et al., Biochem. Biophys. Res. Comm. (1995) 211(3):804; Zhumabayeva et al. (1999) Biotechniques 27:834; Rigas et al. (1986) PNAS USA 83:9591, all of which are expressly incorporated herein by reference). RecA has also been shown to promote rare sequencing searching; see Honigberg et al., PNAS USA 83:9586 (1986), incorporated by reference.

Furthermore, there are a number of systems that have been described for high-throughput manipulation of biological systems; see U.S. Pat. Nos. 5,843,656; 5,856,174; 5,500,356; 5,484,702; 5,759,778; 6,020,187; 5,968,740; 5,962,272; and 6,017,696 and Shepard et al, Nucl. Acid. Res. 25(15):31883 (1997), all of which are expressly incorporated by reference.

This invention describes automation of gene cloning methods that use complementary single-stranded DNA (cssDNA) molecules coated with recombinase proteins to efficiently and specifically target and isolate specific DNA molecules for applications such as DNA cloning; biovalidation of drug targets; DNA modification, including mutagenesis, gene shuffling and evolution; isolation of gene families, orthologs, and paralogs; identification of alternatively spliced isoforms; gene mapping; diagnostic testing for single and multiple nucleotide polymorphisms; differential gene expression and genetic profiling; nucleic acid library production, subtraction and normalization; in situ gene targeting (hybribidization) in cells; in situ gene recombination in cells and animals; high throughput phenotype screening of cells and animals; phenotyping small molecule compounds; screening for pharmaceutical drug regulators; and biovalidation of drugs in transgenic recombinant cells and animals.

The automated, high-throughput technology facilitates the isolation of full-length cDNA clones, identification of functional domains, and validation of the selected sequences. The high-throughput automated analysis of the gene clones (cDNAs, genomic DNA, alternative splice forms, polymorphisms, gene family members) will provide informative analysis of the qualitative differences between expressed genes (gene profiling). Sequence analysis of the isolated cDNAs and genomic DNA allows diagnostic testing for single and multiple nucleotide polymorphisms, loss of heterozygosity (LOH), and other chromosomal abnormalities.

The technology can elucidate differences in gene families and mRNA spliced isoforms, and will provide information on the nature of the mRNA. Libraries of clones obtained at the end of the process will mimic the difference between normal and genetic disorders (or between any differential event). These libraries can be used to screen for genetic signatures and the technology can elucidate precise potential domains of therapeutic intervention within coding sequences of the gene, including catalytic domains (ie, kinases, phosphatases, proteases), protein-protein interaction domains, truncated receptors and soluble receptors.

The methods of the invention can be briefly described as follows. Gene cloning comprising the rapid isolation of cDNA or other nucleic acid clones is facilitated by taking advantage of the catalytic function of the RecA enzyme, an essential component of the bacterial DNA recombination system, which promotes formation of multi-stranded hybrids between ssDNA targeting polynucleotides and homologous double-stranded DNA molecules. The targeting of RecA-coated ssDNAs to homologous sequences at any position in a duplex DNA molecule can produce stable D-loop hybrids. The targeting polynucleotide strands in the D-loop are stable enough to be manipulated by conventional molecular biology procedures. The stability of these multi-stranded hybrid molecules at any position in duplex molecules allows the application of D-loop methods to many different dsDNA substrates, including duplex DNA from cDNA, genomic DNA, or YAC, BAC or PAC libraries. Recombinase coated biotinylated-targeting polynucleotides are targeted to homologous DNA molecules and the targeting polynucleotide: target hybrids are selectively captured on streptavidin-coated magnetic beads. The enriched plasmid population is eluted from the beads and used to transform bacteria or other cells. The resulting colonies are screened by PCR and/or colony hybridization to identify the desired clones. Using this method over 100,000 fold enrichment of the desired clones can be achieved. Furthermore, once the target sequence is cloned, large numbers of variants can be easily generated, again using EHR techniques. These variants can be screened in a wide variety of phenotypic screens, either in the presence or absence of drug candidates.

Examples of automated high throughput applications enabled by EHR technology include rapid gene cloning; mutagenesis, modifications, and evolution of genes; gene mapping; isolation of gene families, gene orthologs, and paralogs; nucleic acid targeting including modified and unmodified DNA and RNA molecules; single and multiple nucleotide polymorphisms diagnostics; loss of heterozygosity (LOH) and other chromosomal aberration diagnostics; recombinase protein and DNA repair assays; nucleic acid library production, subtraction and normalization; analysis of gene expression, genetic quantitation and normalization.

All steps in the gene cloning procedure are amenable to automation. The present invention is directed to the automated gene cloning comprised of the following steps (see also FIG. 1):

9. Library validation and targeting polynucleotide synthesis and purification

10. Clone DNA target capture

11. Transformation and amplification of clone DNA in cells

12. Clone verification (screen for the presence of target sequence by colony picking and PCR)

13. DNA purification

14. Restriction analysis

15. Sequencing

16. Database archiving

Accordingly, the present invention is directed to methods of cloning target nucleic acid sequences. By “cloning” herein is meant the isolation and amplification of a target sequence.

The methods of the invention are directed to the cloning of target nucleic acid sequences. By “target nucleic acid sequence” or “predetermined endogenous DNA sequence” and “predetermined target sequence” refer to polynucleotide sequences contained in a target cell and/or other DNA composition. DNA composition can be a library, or a collection of DNA fragments, for example, a sheared assembly of chromosomal DNA Such sequences include, for example, chromosomal sequences (e.g., structural genes, regulatory sequences including promoters and enhancers, recombinatorial hotspots, repeat sequences, integrated proviral sequences, hairpins, palindromes), episomal or extrachromosomal sequences (e.g., replicable plasmids or viral replication intermediates) including chloroplast and mitochondrial DNA sequences.

The term “regulatory element” is used herein to describe a non-coding sequence which affects the transcription or translation of a gene including, but not limited to, promoter sequences, ribosomal binding sites, transcriptional start and stop sequences, translational start and stop sequences, enhancer or activator sequences, etc. In a preferred embodiment, the regulatory sequences include a promoter and transcriptional start and stop sequence. Promoter sequences can be either constitutive or inducible promoters. The promoters may be either naturally occurring promoters or hybrid promoters. Hybrid promoters, which combine elements of more than one promoter, are also known in the art, and are useful in the present invention. As outlined herein, the target sequence may be a regulatory element.

In general, the target sequence is predetermined. By “predetermined” or “pre-selected” it is meant that the target sequence may be selected at the discretion of the practitioner on the basis of known or predicted sequence information, and is not constrained to specific sites recognized by certain site-specific recombinases (e.g., FLP recombinase or CRE recombinase). In some embodiments, the predetermined endogenous DNA target sequence will be other than a naturally occurring germline DNA sequence (e.g., a transgene, parasitic, mycoplasmal or viral sequence). An exogenous polynucleotide is a polynucleotide which is transferred into a target cell but which has not been replicated in that host cell; for example, a virus genome polynucleotide that enters a cell by fusion of a virion to the cell is an exogenous polynucleotide, however, replicated copies of the viral polynucleotide subsequently made in the infected cell are endogenous sequences (and may, for example, become integrated into a cell chromosome). Similarly, transgenes that are microinjected or transfected into a cell are exogenous polynucleotides, however integrated and replicated copies of the transgene(s) are endogenous sequences.

The term “corresponds to” is used herein to mean that a polynucleotide sequence is homologous (i.e., may be similar or identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. As outlined below, preferably, the homology is at least 70%, preferably 85%, and more preferably 95% identical. Thus, the complementarity between two single-stranded targeting polynucleotides need not be perfect. For illustration, the nucleotide sequence “TATAC” corresponds to a reference sequence “TATAC” and is perfectly complementary to a reference sequence “GTATA”.

The terms “substantially corresponds to” or “substantial identity” or “homologous” as used herein denotes a characteristic of a nucleic acid sequence, wherein a nucleic acid sequence has at least about 70 percent sequence identity as compared to a reference sequence, typically at least about 85 percent sequence identity, and preferably at least about 95 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 18 nucleotides long, typically at least about 30 nucleotides long, and preferably at least about 50 to 100 nucleotides long. “Substantially complementary” as used herein refers to a sequence that is complementary to a sequence that substantially corresponds to a reference sequence. In general, targeting efficiency increases with the length of the targeting polynucleotide portion that is substantially complementary to a reference sequence present in the target DNA.

“Specific hybridization” is defined herein as the formation of hybrids between a targeting polynucleotide (e.g., a polynucleotide of the invention which may include substitutions, deletion, and/or additions as compared to the predetermined target DNA sequence) and a predetermined target DNA, wherein the targeting polynucleotide preferentially hybridizes to the predetermined target DNA such that, for example, at least one discrete band can be identified on a Southern blot of DNA prepared from target cells that contain the target DNA sequence, and/or a targeting polynucleotide in an intact nucleus localizes to a discrete chromosomal location characteristic of a unique or repetitive sequence. In some instances, a target sequence may be present in more than one target polynucleotide species (e.g., a particular target sequence may occur in multiple members of a gene family or in a known repetitive sequence). It is evident that optimal hybridization conditions will vary depending upon the sequence composition and length(s) of the targeting polynucleotide(s) and target(s), and the experimental method selected by the practitioner. Various guidelines may be used to select appropriate hybridization conditions (see Maniatis et al., Molecular Cloning: A Laboratory Manual (1989), 2nd Ed., Cold Spring Harbor, N.Y. and Berger and Cimmel, Methods in Enzymology, Volume 152, Guide to Molecular Cloning Techniques (1987), Academic Press, Inc., San Diego, Calif., which are incorporated herein by reference.

The term “naturally-occurring” as used herein as applied to an object refers to the fact that an object can be found in nature. For example, a polynucleotide sequence that is present in an organism (including viruses) that can be isolated from a source in nature and which has not been intentionally modified by man in the laboratory is naturally-occurring.

A metabolically-active cell is a cell, comprising an intact nucleoid or nucleus, which, when provided nutrients and incubated in an appropriate medium carries out DNA synthesis and RNA for extended periods (e.g., at least 12-24 hours). Such metabolically-active cells are typically undifferentiated or differentiated cells capable or incapable of further cell division (although non-dividing cells many undergo nuclear division and chromosomal replication), although stem cells and progenitor cells are also metabolically-active cells.

In some embodiments, the target sequence is a disease allele. As used herein, the term “disease allele” refers to an allele of a gene that is capable of producing a recognizable disease. A disease allele may be dominant or recessive and may produce disease directly or when present in combination with a specific genetic background or pre-existing pathological condition. A disease allele may be present in the gene pool or may be generated de novo in an individual by somatic mutation. For example and not limitation, disease to alleles include: activated oncogenes, a sickle cell anemia allele, a Tay-Sachs allele, a cystic fibrosis allele, a Lesch-Nyhan allele, a retinoblastoma-susceptibility allele, a Fabry's disease allele, and a Huntington's chorea allele. As used herein, a disease allele encompasses both alleles associated with human diseases and alleles associated with recognized veterinary diseases. For example, the F508 CFTR allele in a human disease allele which is associated with cystic fibrosis in North Americans.

Recombinase

The methods of the invention comprise providing an enhanced homologous recombination (EHR) composition comprising a recombinase. By “recombinase” herein is meant a protein that, when included with an exogenous targeting polynucleotide, provide a measurable increase in the recombination frequency and/or localization frequency between the targeting polynucleotide and an endogenous predetermined DNA sequence. Thus, in a preferred embodiment, increases in recombination frequency from the normal range of 10⁻⁸-10⁻⁴, to 10⁻⁴-10⁰, preferably 10⁻³-10⁰, and most preferably 10⁻²-10⁰, may be achieved.

In the present invention, recombinase refers to a family of RecA-like recombination proteins all having essentially all or most of the same functions, particularly: (i) the recombinase protein's ability to properly bind to and position targeting polynucleotides on their homologous targets and (ii) the ability of recombinase protein/targeting polynucleotide complexes to efficiently find and bind to complementary endogenous sequences. The best characterized RecA protein is from the bacterium E. coli. In addition to the wild-type protein a number of mutant RecA proteins have been identified (e.g., RecA803; see Madiraju et al., PNAS USA 85(18):6592 (1988); Madiraju et al, Biochem. 31:10529 (1992); Lavery et al., J. Biol. Chem. 267:20648 (1992)). Further, many organisms have RecA-like recombinases with strand-transfer activities (e.g., Fugisawa et al., (1985) Nucl. Acids Res. 13: 7473; Hsich et al., (1986) Cell 44: 885; Hsieh et al., (1989) J. Biol. Chem. 264: 5089; Fishel et al., (1988) Proc. Natl. Acad. Sci. (USA) 85: 3683; Cassuto et al., (1987) Mol. Gen. Genet. 208: 10; Ganea et al., (1987) Mol. Cell Biol. 7: 3124; Moore et al., (1990) J. Biol. Chem. 19: 11108; Keene et al., (1984) Nucl. Acids Res. 12: 3057; Kimeic, (1984) Cold Spring Harbor Symp. 48: 675; Kmeic, (1986) Cell 44: 545; Kolodner et al., (1987) Proc. Natl. Acad. Sci. USA 84: 5560; Sugino et al., (1985) Proc. Natl. Acad. Sci. USA 85: 3683; Halbrook et al., (1989) J. Biol. Chem. 264: 21403; Eisen et al., (1988) Proc. Natl. Acad. Sci. USA 85: 7481; McCarthy et al., (1988) Proc. Natl. Acad. Sci. USA 85: 5854; Lowenhaupt et al., (1989) J. Biol. Chem. 264: 20568, which are incorporated herein by reference). Examples of such recombinase proteins include, for example but not limited to: RecA, RecA803, UvsX, and other RecA mutants and RecA-like recombinases (Roca, A. I. (1990) Crit. Rev. Biochem. Molec. Biol. 25: 415), sep1 (Kolodner et al. (1987) Proc. Natl. Acad. Sci. (U.S.A.) 84:5560; Tishkoff et al. Molec. Cell. Biol. 11:2593), RuvC (Dunderdale et al. (1991) Nature 354: 506), DST2, KEM1, XRN1 (Dykstra et al. (1991) Molec. Cell. Biol. 11:2583), STP/DST1 (Clark et al. (1991) Molec. Cell. Biol. 11:2576), HPP-1 (Moore et al. (1991) Proc. Natl. Acad. Sci. (U.S.A.) 88:9067), other target recombinases (Bishop et al. (1992) Cell 69: 439; Shinohara et al. (1992) Cell 69: 457); incorporated herein by reference). RecA may be purified from E. coli strains, such as E. coli strains JC12772 and JC15369 (available from A. J. Clark and M. Madiraju, University of California-Berkeley, or purchased commercially). These strains contain the recA coding sequences on a “runaway” replicating plasmid vector (present at a high copy number in the cell). The RecA803 protein is a high-activity mutant of wild-type RecA. The art teaches several examples of recombinase proteins, for example, from Drosophila, yeast, plant, human, and non-human mammalian cells, including proteins with biological properties similar to RecA (i.e., RecA-like recombinases), such as Rad51, Rad57, dmel from mammals and yeast, and Pk-rec (see Rashid et al., Nucleic Acid Res. 25(4):719 (1997), hereby incorporated by reference). In addition, the recombinase may actually be a complex of proteins, i.e. a “recombinosome”. In addition, included within the definition of a recombinase are portions or fragments of recombinases which retain recombinase biological activity, as well as variants or mutants of wild-type recombinases which retain biological activity, such as the E. coli RecA803 mutant with enhanced recombinase activity.

In a preferred embodiment, RecA or Rad51 is used. For example, RecA protein is typically obtained from bacterial strains that overproduce the protein: wild-type E. Coli RecA protein and mutant RecA803 protein may be purified from such strains. Alternatively, RecA protein can also be purchased from, for example, Pharmacia (Piscataway, N.J.) or Boehringer Mannheim (Indianapolis, Ind.).

RecA proteins, and their homologs, form a nucleoprotein filament when they coat a single-stranded DNA molecule. In this nucleoprotein filament, one monomer of RecA protein is bound to about 3 nucleotides. This ability of RecA to coat single-stranded DNA is essentially sequence independent, although particular sequences favor initial loading of RecA onto a polynucleotide (e.g., nucleation sequences). The nucleoprotein filament(s) can be formed on essentially any DNA molecule and can be formed in cells (e.g., mammalian cells), forming complexes with both single-stranded and double-stranded DNA, although the loading conditions for dsDNA are somewhat different than for ssDNA.

Targeting Polynucleotides

The recombinase is combined with targeting polynucleotides as is more fully outlined below. By “nucleic acid” or “oligonucleotide” or “polynucleotide” or grammatical equivalents herein means at least two nucleotides covalently linked together. A nucleic acid of the present invention will generally contain phosphodiester bonds, although in some cases nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986), phosphorothioate, phosphorodithioate, O-methylphophoroamidite linkages (see Eckstein, Oligonucleotides and Analogues: A Practical Approach, Oxford University Press), and peptide nucleic acid (PNA) backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). These modifications of the ribose-phosphate backbone or bases may be done to facilitate the addition of other moieties such as chemical constituents, including 2′ O-methyl and 5′ modified substituents, as discussed below, or to increase the stability and half-life of such molecules in physiological environments.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273: 1386 (1996) and Yoon et al., PNAS USA 93:2071 (1996), both of which are hereby incorporated by reference.

In general, the targeting polynucleotides may comprise any number of structures, as long as the changes do not substantially effect the functional ability of the targeting polynucleotide to result in homologous recombination. For example, recombinase coating of alternate structures should still be able to occur.

By “targeting polynucleotides” herein is meant the polynucleotides used to clone or alter the target nucleic acids as described herein. Targeting polynucleotides are generally ssDNA or dsDNA, most preferably two complementary single-stranded DNAs.

Targeting polynucleotides are generally at least about 5 to 2000 nucleotides long, preferably about 12 to 200 nucleotides long, at least about 200 to 500 nucleotides long, more preferably at least about 500 to 2000 nucleotides long, or longer; however, as the length of a targeting polynucleotide increases beyond about 20,000 to 50,000 to 400,000 nucleotides, the efficiency or transferring an intact targeting polynucleotide into the cell decreases. The length of homology may be selected at the discretion of the practitioner on the basis of the sequence composition and complexity of the predetermined endogenous target DNA sequence(s) and guidance provided in the art, which generally indicates that 1.3 to 6.8 kilobase segments of homology are preferred when non-recombinase mediated methods are utilized (Hasty et al. (1991) Molec. Cell. Biol. 11: 5586; Shulman et al. (1990) Molec. Cell. Biol. 10: 4466, which are incorporated herein by reference).

Targeting polynucleotides have at least one sequence that substantially corresponds to, or is substantially complementary to, the target nucleic acid, i.e. the predetermined endogenous DNA sequence (i.e., a DNA sequence of a polynucleotide located in a target cell, such as a chromosomal, mitochondrial, chloroplast, viral, extra chromosomal, or mycoplasmal polynucleotide). By “corresponds to” herein is meant that a polynucleotide sequence is homologous (i.e., may be similar or identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence can hybridize to all or a portion of a reference polynucleotide sequence. Thus, one of the complementary single stranded targeting polynucleotides is complementary to one strand of the endogenous target sequence (i.e. Watson) and corresponds to the other strand of the endogenous target sequence (i.e. Crick). Thus, the complementarity between two single-stranded targeting polynucleotides need not be perfect. For illustration, the nucleotide sequence “TATAC” corresponds to a reference sequence “TATAC” and is perfectly complementary to a reference sequence “GTATA”.

The terms “substantially corresponds to” or “substantial identity” or “homologous” as used herein denotes a characteristic of a nucleic acid sequence, wherein a nucleic acid sequence has at least about 50 percent sequence identity as compared to a reference sequence, typically at least about 70 percent sequence identity, and preferably at least about 85 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 18 nucleotides long, typically at least about 30 nucleotides long, and preferably at least about 50 to 100 nucleotides long. “Substantially complementary” as used herein refers to a sequence that is complementary to a sequence that substantially corresponds to a reference sequence. In general, targeting efficiency increases with the length of the targeting polynucleotide portion that is substantially complementary to a reference sequence present in the target DNA.

These corresponding/complementary sequences are referred to herein as “homology clamps”, as they serve as templates for homologous pairing with the target sequence(s). Thus, a “homology clamp” is a portion of the targeting polynucleotide that can specifically hybridize to a portion of a target sequence. “Specific hybridization” is defined herein as the formation of hybrids between a targeting polynucleotide (e.g., a polynucleotide of the invention which may include substitutions, deletion, and/or additions as compared to the predetermined target nucleic acid sequence) and a target nucleic acid, wherein the targeting polynucleotide preferentially hybridizes to the target nucleic acid such that, for example, at least one discrete band can be identified on a Southern blot of nucleic acid prepared from target cells that contain the target nucleic acid sequence, and/or a targeting polynucleotide in an intact nucleus localizes to a discrete chromosomal location characteristic of a unique or repetitive sequence. It is evident that optimal hybridization conditions will vary depending upon the sequence composition and length(s) of the targeting polynucleotide(s) and target(s), and the experimental method selected by the practitioner. Various guidelines may be used to select appropriate hybridization conditions (see, Maniatis et al., Molecular Cloning: A Laboratory Manual (1989), 2nd Ed., Cold Spring Harbor, N.Y. and Berger and Kimmel, Methods in Enzymology, Volume 152, Guide to Molecular Cloning Techniques (1987), Academic Press, Inc., San Diego, Calif.), which are incorporated herein by reference. Methods for hybridizing a targeting polynucleotide to a discrete chromosomal location in intact nuclei are known in the art, see for example WO 93/05177 and Kowalczykowski and Zarling (1994) in Gene Targeting, Ed. Manuel Vega.

In targeting polynucleotides, such homology clamps are typically located at or near the 5′ or 3′ end, preferably homology clamps are internal or located at each end of the polynucleotide (Berinstein et al. (1992) Molec, Cell. Biol. 12: 360, which is incorporated herein by reference). Without wishing to be bound by any particular theory, it is believed that the addition of recombinases permits efficient gene targeting with targeting polynucleotides having short (i.e., about 10 to 1000 base pair long) segments of homology, as well as with targeting polynucleotides having longer segments of homology.

Therefore, it is preferred that targeting polynucleotides of the invention have homology clamps that are highly homologous to the target endogenous nucleic acid sequence(s). Typically, targeting polynucleotides of the invention have at least one homology clamp that is at least about 18 to 35 nucleotides long, and it is preferable that homology clamps are at least about 20 to 100 nucleotides long, and more preferably at least about 100-500 nucleotides long, although the degree of sequence homology between the homology clamp and the targeted sequence and the base composition of the targeted sequence will determine the optimal and minimal clamp lengths (e.g., G-C rich sequences are typically more thermodynamically stable and will generally require shorter clamp length). Therefore, both homology clamp length and the degree of sequence homology can only be determined with reference to a particular predetermined sequence, but homology clamps generally must be at least about 10 nucleotides long and must also substantially correspond or be substantially complementary to a predetermined target sequence. Preferably, a homology clamp is at least about 10, and preferably at least about 50 nucleotides long and is substantially identical to or complementary to a predetermined target sequence.

In a preferred embodiment, two substantially complementary targeting polynucleotides are used. In one embodiment, the targeting polynucleotides form a double stranded hybrid, which may be coated with recombinase, although when the recombinase is RecA, the loading conditions may be somewhat different from those used for single stranded nucleic acids.

In a preferred embodiment, two substantially complementary single-stranded targeting polynucleotides are used. The two complementary single-stranded targeting polynucleotides are usually of equal length, although this is not required. However, as noted below, the stability of the four strand hybrids of the invention is putatively related, in part, to the lack of significant unhybridized single-stranded nucleic acid, and thus significant unpaired sequences are not preferred. Furthermore, as noted above, the complementarity between the two targeting polynucleotides need not be perfect. The two complementary single-stranded targeting polynucleotides are simultaneously or contemporaneously introduced into a target cell harboring a predetermined endogenous target sequence, generally with at lease one recombinase protein (e.g., RecA). Under most circumstances, it is preferred that the targeting polynucleotides are incubated with RecA or other recombinase prior to introduction into a target cell, so that the recombinase protein(s) may be “loaded” onto the targeting polynucleotide(s), to coat the nucleic acid, as is described below. Incubation conditions for such recombinase loading are described infra, and also in U.S. Ser. No. 07/755,462, filed Sep. 4, 1991; U.S. Ser. No. 07/910,791, filed Jul. 9, 1992; and U.S. Ser. No. 07/520,321, filed May 7, 1990, each of which is incorporated herein by reference. A targeting polynucleotide may contain a sequence that enhances the loading process of a recombinase, for example a RecA loading sequence is the recombinogenic nucleation sequence poly[d(A-C)], and its complement, poly[d(G-T)]. The duplex sequence poly[d(A-C) d(G-T)n, where n is from 5 to 25, is a middle repetitive element in target DNA.

There appears to be a fundamental difference in the stability of RecA-protein-mediated D-loops formed between one single-stranded DNA (ssDNA) targeting polynucleotide hybridized to negatively supercoiled DNA targets in comparison to relaxed or linear duplex DNA targets. Internally located dsDNA target sequences on relaxed linear DNA targets hybridized by ssDNA targeting polynucleotides produce single D-loops, which are unstable after removal of RecA protein (Adzuma, Genes Devel. 6:1679 (1992); Hsieh et al, PNAS USA 89:6492 (1992); Chiu et al., Biochemistry 32:13146 (1993)). This targeting polynucleotide instability of hybrids formed with linear duplex DNA targets is most probably due to the incoming ssDNA targeting polynucleotide W-C base pairing with the complementary DNA strand of the duplex target and disrupting the base pairing in the other DNA strand. The required high free-energy of maintaining a disrupted DNA strand in an unpaired ssDNA conformation in a protein-free single-D-loop apparently can only be compensated for either by the stored free energy inherent in negatively supercoiled DNA targets or by base pairing initiated at the distal ends of the joint DNA molecule, allowing the exchanged strands to freely intertwine.

However, the addition of a second complementary ssDNA to the three-strand-containing single-D-loop stabilizes the deproteinized hybrid joint molecules by allowing W-C base pairing of the targeting polynucleotide with the displaced target DNA strand. The addition of a second RecA-coated complementary ssDNA (cssDNA) strand to the three-strand containing single D-loop stabilizes deproteinized hybrid joints located away from the free ends of the duplex target DNA (Sena & Zarling, Nature Genetics 3:365 (1993); Revet et al. J. Mol. Biol. 232:779 (1993); Jayasena and Johnston, J. Mol. Bio. 230:1015 (1993)). The resulting four-stranded structure, named a double D-loop by analogy with the three-stranded single D-loop hybrid has been shown to be stable in the absence of RecA protein. This stability likely occurs because the restoration of W-C base pairing in the parental duplex would require disruption of two W-C base pairs in the double-D-loop (one W-C pair in each heteroduplex D-loop). Since each base-pairing in the reverse transition (double-D-loop to duplex) is less favorable by the energy of one W-C base pair, the pair of cssDNA targeting polynucleotides is thus kinetically trapped in duplex DNA targets in stable hybrid structures. The stability of the double-D loop joint molecule within internally located targeting polynucleotide:target hybrids is an intermediate stage prior to the progression of the homologous recombination reaction to the strand exchange phase. The double D-loop permits isolation of stable multi-stranded DNA recombination intermediates.

The invention may in some instances be practiced with individual targeting polynucleotides that do not comprise part of a complementary pair. In each case, a targeting polynucleotide is introduced into a target cell simultaneously or contemporaneously with a recombinase protein, typically in the form of a recombinase coated targeting polynucleotide as outlined herein (i.e., a polynucleotide pre-incubated with recombinase wherein the recombinase is non-covalently bound to the polynucleotide; generally referred to in the art as a nucleoprotein filament). Alternatively, the use of a single targeting polynucleotide may be done in gene chip applications, as outlined below.

Thus, compositions of the present invention preferably include, in addition to a recombinase, a first and a second targeting polynucleotide. As noted herein, either the first or the second polynucleotide comprises a fragment of a target nucleic acid, although in some instances it may comprise the entire target nucleic acid.

In a preferred embodiment, the first polynucleotide is an expressed sequence tag (EST). As will be appreciated by those in the art, there are a wide variety of ESTs known, either publicly or privately. By using an EST as the first polynucleotide, the full-length gene may be cloned as outlined herein. Alternatively the polynucleotide can be any partial gene sequence.

As will be appreciated by those in the art, there are a variety of ways to generate targeting polynucleotides. In one embodiment, for example when an EST sequence is to serve as the targeting polynucleotide, primers are generated as outlined herein; alternatively, the polynucleotides can be made directly, using known synthetic techniques. Additionally, for large targeting polynucleotides, plasmids are engineered to contain an appropriately sized gene sequence with a deletion or insertion in the gene of interest and at least one flanking homology clamp, which substantially corresponds or is substantially complementary to an endogenous target DNA sequence. Vectors containing a targeting polynucleotide sequence are typically grown in E. coli and then isolated using standard molecular biology methods. Alternatively, targeting polynucleotides may be prepared in single-stranded form by oligonucleotide synthesis methods, which may first require, especially with larger targeting polynucleotides, formation of subfragments of the targeting polynucleotide, typically followed by splicing of the subfragments together, typically by enzymatic ligation or by PCR. In general, as will be appreciated by those in the art, targeting polynucleotides may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence.

Separation Moieties

In a preferred embodiment, in addition to the recombinase and targeting polynucleotides, the EHR compositions of the invention comprise a separation moiety. By “separation moiety” or “purification moiety” or grammatical equivalents herein is meant a moiety which may be used to purify or isolate the nucleic acids, including the targeting polynucleotides, the targeting polynucleotide:target sequence complex, or the target sequence. As will be appreciated by those in the art, the separation moieties may comprise any number of different entities, including, but not limited to, haptens such as chemical moieties, epitope tags, binding partners, or unique nucleic acid sequences; basically anything that can be used to isolate or separate a targeting polynucleotide:target sequence complex from the rest of the nucleic acids present.

For example, in a preferred embodiment, the separation moiety is a binding partner pair, such as biotin, such that biotinylated targeting targeting polynucleotides are made, and streptavidin or avidin columns or beads plates (particularly magnetic beads as described herein) can be used to isolate the targeting targeting polynucleotide:target sequence complex.

In a preferred embodiment, the targeting polynucleotides are biotinylated. Partial cDNA or EST-size fragments, prepared as biotinylated-ssDNA targeting polynucleotides, are used to target cDNA or gDNA libraries, or some other composition containing the target DNA for the formation of stable biotinylated-targeting polynucleotide:target hybrids. Oligonucleotides (generally 20-30 bases) that were complementary to the target nucleic acid or Expressed Sequence Tag (EST) sequence are designed using known techniques, including the Primer3 Software Program. These primers are used in PCR reactions to screen DNA compositions containing the target DNA (e.g. cDNA libraries) for presence of the desired target. The reaction products are analyzed by agarose gel electrophoresis. In case of multiple bands, the correct PCR product is purified using any of available gel purification procedures (e.g. Qiagen's column based protocol, or Promega's MagneSil magnetic bead based protocol). Internally-labeled, biotinylated DNA fragments or targeting polynucleotides (generally 200-1000 bp) are then synthesized by PCR in the presence of biotin-dATP and dATP at a ratio of 1:3, dTTP, dCTP, and dGTP, from either the purified PCR product template, or directly from the source (any composition containing the target DNA). Alternatively, 5′-labeled biotinylated targeting polynucleotides are generated by incorporation of a 5′-biotinylated primer into the DNA fragment during PCR. The product can be purified directly, or it can be run on a gel, and the correct band cut and purified. The targeting polynucleotides are purified using any of available PCR clean up procedures (e.g. G-50 or G-25 spin columns (Amersham-Pharmacia), Promega MagneSil magnetic bead based protocols, Qiagen QiaQuick) to remove unincorporated nucleotides and primers. The concentration of the purified targeting nucleotides is determined by reading the absorbance at 260 nm in a plate reader. The targeting polynucleotides are diluted to 25 ng/ul with TE′ (10 mM Tris-HCl, pH 7.5, 0.1 mM EDTA).

In a preferred embodiment, the separation moiety is an epitope tag. Suitable epitope tags include myc (for use with the commercially available 9E10 antibody), the BSP biotinylation target sequence of the bacterial enzyme BirA, flu tags, lacZ, and GST.

Alternatively, the separation moiety may be a separation sequence that is a unique oligonucleotide sequence which serves as a targeting polynucleotide target site to allow the quick and easy isolation of the complex; for example using an affinity-type column.

Gene Families

In a preferred embodiment, the first polynucleotide is a consensus homology motif tag as outlined in WO 99/37755, hereby expressly incorporated by reference. In this embodiment, a consensus sequence can be used to clone members of a gene family that share a consensus sequence. By “homology motif tag” or “protein consensus sequence” herein is meant an amino acid consensus sequence of a gene family. By “consensus nucleic acid sequence” herein is meant a nucleic acid that encodes a consensus protein sequence of a functional domain of a gene family. In addition, “consensus nucleic acid sequence” can also refer to cis sequences that are non-coding but can serve a regulatory or other role. As outlined below, generally a library of consensus nucleic acid sequences are used, that comprises a set of degenerate nucleic acids encoding the protein consensus sequence. A wide variety of protein consensus sequences for a number of gene families are known. A “gene family” therefore is a set of genes that encode proteins that contain a functional domain for which a consensus sequence can be identified. However, in some instances, a gene family includes non-coding sequences; for example, consensus regulatory regions can be identified. For example, gene family/consensus sequences pairs are known for the G-protein coupled receptor family, the AAA-protein family, the bZIP transcription factor family, the mutS family, the recA family, the Rad51 family, the dmel family, the recF family, the SH2 domain family, the Bcl-2 family, the single-stranded binding protein family, the TFIID transcription family, the TGF-beta family, the TNF family, the XPA family, the XPG family, actin binding proteins, bromodomain GDP exchange factors, MCM family, ser/thr phosphatase family, etc.

As will be appreciated by those in the art, the proteins of the gene families generally do not contain the exact consensus sequences; generally consensus sequences are artificial sequences that represent the best comparison of a variety of sequences. The actual sequence that corresponds to the functional sequence within a particular protein is termed a “consensus functional domain” herein; that is, a consensus functional domain is the actual sequence within a protein that corresponds to the consensus sequence. A consensus functional domain may also be a “predetermined endogenous DNA sequence” (also referred to herein as a “predetermined target sequence”) that is a polynucleotide sequence contained in a target cell. Such sequences can include, for example, chromosomal sequences (e.g., structural genes, regulatory sequences including promoters and enhancers, recombinatorial hotspots, repeat sequences, integrated proviral sequences, hairpins, palindromes), episomal or extrachromosomal sequences (e.g., replicable plasmids or viral replication intermediates) including chloroplast and mitochondrial DNA sequences. By “predetermined” or “pre-selected” it is meant that the consensus functional domain target sequence may be selected at the discretion of the practitioner on the basis of known or predicted sequence information, and is not constrained to specific sites recognized by certain site-specific recombinases (e.g., FLP recombinase or CRE recombinase). In some embodiments, the predetermined endogenous DNA target sequence will be other than a naturally occurring germline DNA sequence (e.g., a transgene, parasitic, mycoplasmal or viral sequence).

In a preferred embodiment, the gene family is the G-protein coupled receptor family, which has only 900 identified members, includes several subfamilies and may include over 13,2000 genes. In a preferred embodiment, the G-protein coupled receptors are from subfamily 1 and are also called R7G proteins. They are an extensive group of receptors that recognize hormones, neurotransmitters, odorants and light and transduce extracellular signals by interaction with guanine (G) nucleotide-binding proteins. The structure of all these receptors is thought to be virtually identical, and they contain seven hydrophobic regions, each of which putatively spans the membrane. The N-terminus is extracellular and is frequently glycosylated, and the C-terminus is cytoplasmic and generally phosphorylated. Three extracellular loops alternate with three cytoplasmic loops to link the seven transmembrane regions. G-protein coupled receptors include, but are not limited to: the class A rhodopsin first subfamily, including amine (acetylcholine (muscarinic), adrenoceptors, domamine, histamine, serotonin, octopamine), peptides (angiotensin, bombesin, bradykinin, C5a anaphylatoxin, Fmet-leu-phe, interleukin-8, chemokine, CCK, endothelin, mealnocortin, neuropeptide Y, neurotensin, opioid, somatostatin, tachykinin, thrombin, vasopressin-like, galanin, proteinase activated), hormone proteins (follicle stimulating hormone, lutropin-choriogonadotropic hormone, thyrotropin), rhodopsin (vertebrate), olfactory (olfactory type 1-11, gustatory), prostanoid (prostaglandin, prostacyclin, thromboxane), nucleotide (adenosine, purinoceptors), cannabis, platelet activating factor, gonadotropin-releasing hormone (gonadotropin releasing hormone, thyrotropin-releasing hormone, growth hormone secretagogue), melatonin, viral proteins, MHC receptor, Mas proto-oncogene, EBV-induced and glucocorticoid induced; the class B secretin second subfamily, including calcitonin, corticotropin releasing factor, gastric inhibitory peptide, glucagon, growth hormone releasing hormone, parathyroid hormone, secretin, vasoactive intestinal polypeptide, and diuretic hormone; the class C metabotropic glutamate third subfamily, including metabrotropic glutamate and extracellular calcium-sensing agents; and the class D pheromone fourth subfamily.

Because of the large number of family members, these large classes of GPCRs can be further subdivided into subfamilies where metabotropic is from class C; calcitonin, glucagon, vasoactive and parathyroid are from class B; and acetylcholine, histamine angiotensin, 2- and -adrenergic are from class A. From each subfamily small protein consensus sequences can be derived from sequence alignments. Using the protein consensus sequence, degenerate targeting polynucleotides are made to encode the protein consensus sequence, as is well known in the art. The protein sequence is encoded by DNA triplets, which are deduced using standard tables. In some cases additional degeneracy is used to enable production in one oligonucleotide synthesis. In many cases motifs were chosen to minimize degeneracy. In addition, the consensus sequences may be designed to facilitate amplification of neighboring sequences. This can utilize two motifs as indicated by faithful or error prone amplification. Alternatively outside sequences can be used as is indicated using vector sequence. In addition degenerate oligos can be synthesized and used directly in the procedure without amplification.

In addition to the first subfamily of G-protein coupled receptors, there is a second subfamily encoding receptors that bind peptide hormones that do not show sequence similarity to the first R7G subfamily. All the characterized receptors in this subfamily are coupled to G-proteins that activate both adenylyl cyclase and the phosphatidylinositol-calcium pathway. However, they are structurally similar; like classical R7G proteins they putatively contain seven transmembrane regions, a glycosylated extracellular N-terminus and a cytoplasmic C-terminus. Known receptors in this subfamily are encoded on multiple exons, and several of these genes are alternatively spliced to yield functionally distinct products. The N-terminus contains five conserved cysteine residues putatively important in disulfide bonds. Known G-protein coupled receptors in this subfamily are listed above.

In addition to the first and second subfamilies of G-protein coupled receptors, there is a third subfamily encoding receptors that bind glutamate and calcium but do not show sequence similarity to either of the other subfamilies. Structurally, this subfamily has signal sequences, very large hydrophobic extracellular regions of about 540 to 600 amino acids that contain 17 conserved cysteines (putatively involved in disulfides), a region of about 250 residues that appear to contain seven transmembrane domains, and a C-terminal cytoplasmic domain of variable length (50 to 350 residues). Known G-protein coupled receptors of this subfamily are listed above.

In a preferred embodiment, the gene family is the bZIP transcription factor family. This eukaryotic gene family encodes DNA binding transcription factors that contain a basic region that mediates sequence specific DNA binding, and a leucine zipper, required for dimerization. The bZIP family includes, but is not limited to, AP-1, ATF, CREB, CREM, FOS, FRA, GBF, GCN4, HBP, JUN, MET4, OCS1, OP, TAF1, XBP1, and YBBO.

In a preferred embodiment, the gene family is involved in DNA mismatch repair, such as mutL, hexB and PMS1. Members of this family include, but are not limited to, MLH1, PMS1, PMS2, HexB and MulL. The protein consensus sequence is G-F-R-G-E-A-L.

In a preferred embodiment, the gene family is the mutS family, also involved in mismatch repair of DNA, directed to the correction of mismatched base pairs that have been missed by the proofreading element of the DNA polymerase complex. mutS gene family members include, but are not limited to, MSH2, MSH3, MSH6 and MutS.

In a preferred embodiment, the gene family is the recA family. The bacterial recA is essential for homologous recombination and recombinatorial repair of DNA damage. RecA has many activities, including the formation of nucleoprotein filaments, binding to single stranded and double stranded DNA, binding and hydrolyzing ATP, recombinase activity and interaction with LexA causing LexA activation and auto catalytic cleavage. RecA family members include those from E. coli, drosophila, human, lily, etc. specifically including but not limited to, E. coli RecA, Rec1, Rec2, Rad51, Rad51 B, Rad51C, Rad51D, Rad51E, XRCC2 and DMC1.

In a preferred embodiment, the gene family is the recF family. The prokaryotic RecF protein is a single-stranded DNA binding protein that also putatively binds ATP. RecF is involved in DNA metabolism; it is required for recombinatorial DNA repair and for induction of the SOS response. RecF is a protein of about 350 to 370 amino acid residues; there is a conserved ATP-binding site motif ‘A’ in the N-terminal section of the protein as well as two other conserved regions, one located in the central section and the other in the C-terminal section.

In a preferred embodiment, the gene family is the Bcl-2 family. Programmed cell death (PCD), or apoptosis, is induced by events such as growth factor withdrawal and toxins. It is generally controlled by regulators, which have either an inhibitory effect (i.e. anti-apoptotic) or block the protective effect of inhibitors (pro-apoptotic). Many viruses have found a way of countering defensive apoptosis by encoding their own anti-apoptotic genes thereby preventing their target cells from dying too soon.

All proteins belonging to the Bcl-2 family contain at least one of a BH1, BH2, BH3 or BH4 domain. All anti-apoptotic proteins contain BH1 and BH2 domains, some of them contain an additional N-terminal BH4 domain (such as Bcl-2, Bcl-x(L), Bcl-W, etc.), which is generally not found in pro-apoptotic proteins (with the exception of Bcl-x(S). Generally all pro-apoptotic proteins contain a BH3 domain (except for Bad), thought to be crucial for the dimerization of the proteins with other Bcl-2 family members and crucial for their killing activity. In addition, some of the pro-apoptotic proteins contain BH1 and BH2 domains (such as Bax and Bak). The BH3 domain is also present in some anti-apoptosis proteins, such as Bcl-2 and Bcl-x(L). Known Bcl-2 proteins include, but are not limited to, Bcl-2, Bcl-x(L), Bcl-W, Bcl-x(S), Bad, Bax, and Bak.

In a preferred embodiment, the gene family is the site-specific recombinase family. Site-specific recombination plays an important role in DNA rearrangement in prokaryotic organisms. Two types of site-specific recombination are known to occur: a) recombination between inverted repeats resulting in the reversal of a DNA segment; and b) recombination between repeat sequences on two DNA molecules resulting in their co-integration, or between repeats on one DNA molecule resulting the excision of a DNA fragment. Site-specific recombination is characterized by a strand exchange mechanism that requires no DNA synthesis or high-energy cofactor; the phosphodiester bond energy is conserved in a phospho-protein linkage during strand cleavage and re-ligation.

Two unrelated families of recombinases are currently known. The first, called the “phage integrase” family, groups a number of bacterial, phage and yeast plasmid enzymes. The second, called the “resolvase” family, groups enzymes which share the following structural characteristics: an N-terminal catalytic and dimerization domain that contains a conserved serine residue involved in the transient covalent attachment to DNA, and a C-terminal helix-turn-helix DNA-binding domain.

In a preferred embodiment, the gene family is the single-stranded binding protein family. The E. coli single-stranded binding protein (ssb), also known as the helix-destabilizing protein, is a protein of 177 amino acids. It binds tightly as a homotetramer to a single-stranded DNA (ssDNA) and plays an important role in DNA replication, recombination and repair. Members of the ssb family include, but are not limited to, E. Coli ssb and eukaryotic RPA proteins.

In a preferred embodiment, the gene family is the TFIID transcription family. Transcription factor TFIID (or TATA-binding protein, TBP), is a general factor that plays a major role in the activation of eukaryotic genes transcribed by RNA polymerase II. TFIID binds specifically to the TATA box promoter element, which lies close to the position of transcription initiation. There is a remarkable degree of sequence conservation of a C-terminal domain of about 180 residues in TFIID from various eukaryotic sources. This region is necessary and sufficient for TATA box binding. The most significant structural feature of this domain is the presence of two conserved repeats of a 77 amino-acid region.

In a preferred embodiment, the gene family is the TGF-b family. Transforming growth factor-(TGF-b) is a multifunctional protein that controls proliferation, differentiation and other functions in many cell types. TGF-b-1 is a protein of 112 amino acid residues derived by proteolytic cleavage from the C-terminal portion of the precursor protein. Members of the TGF-b family include, but are not limited to, the TGF-1-3 subfamily (including TGF1, TGF2, and TGF3); the BMP3 subfamily (BM3B, BMP3); the BMP5-8 subfamily (BM8A, BMP5, BMP6, BMP7, and BMP8); and the BMP 2 & 4 subfamily (BMP2, BMP4, DECA).

In a preferred embodiment, the gene family is the TNF family. A number of cytokines can be grouped into a family on the basis of amino acid sequence, as well as structural and functional similarities. These include (1) tumor necrosis factor (TNF), also known as cachectin or TNF-a, which is a cytokine with a wide variety of functions. TNF-a can cause cytolysis of certain tumor cell lines; it is involved in the induction of cachexia; it is a potent pyrogen, causing fever by direct action or by stimulation of interleukin-1 secretion; and it can stimulate cell proliferation and induce cell differentiation under certain conditions; (2) lymphotoxin-a (LT-a) and lymphotoxin-b (LT-b), two related cytokines produced by lymphocytes and which are cytotoxic for a wide range of tumor cells in vitro and in vivo; (3) T cell antigen gp39 (CD40L), a cytokine that seems to be important in B-cell development and activation; (4) CD27L, a cytokine that plays a role in T-cell activation; it induces the proliferation of co-stimulated T cells and enhances the generation of cytolytic T cells; (5) CD30L, a cytokine that induces proliferation of T-cells; (6) FASL, a cytokine involved in cell death; (8) 4-1BBL, an inducible T cell surface molecule that contributes to T-cell stimulation; (9) OX40L, a cytokine that co-stimulates T cell proliferation and cytokine production; and (10), TNF-related apoptosis inducing ligand (TRAIL), a cytokine that induces apoptosis.

In a preferred embodiment, the gene family is the XPA family. Xeroderma pigmentosa (XP) is a human autosomal recessive disease, characterized by a high incidence of sunlight-induced skin cancer. Skin cells associated with this condition are hypersensitive to ultraviolet light, due to defects in the incision step of DNA excision repair. There are a minimum of 7 genetic complementation groups involved in this disorder: XPA to XPG. XPA is the most common form of the disease and is due to defects in a 30 kD nuclear protein called XPA or (XPAC). The sequence of XPA is conserved from higher eukaryotes to yeast (gene RAD14). XPA is a hydrophilic protein of 247 to 296 amino acid residues that has a C4-type zinc finger motif in its central section.

In a preferred embodiment, the gene family is the XPG family. The defect in XPG can be corrected by a 133 kD nuclear protein called XPG (or XPGC). Members of the XPG family include, but are not limited to, FEN1, XPG, RAD2, EXO1, and DIN7.

In a preferred embodiment, the present invention finds use not only in cloning the exact match to a targeting polynucleotide, but also in the isolation of new members of gene families. As is generally described herein and in related applications, the use of HMT filaments (i.e. consensus homology clamps preferably containing a purification tag such as biotin, digoxigenin, or another purification method such as the use of a RecA antibody), allows the identification of new genes within the gene family. Once identified, the new genes can be cloned, sequenced and the protein gene products purified. As will be appreciated by those in the art, the functional importance of the new genes can be assessed in a number of ways, including functional studies on the protein level, phenotypic screening, as well as the generation of “knock out” or genetically altered animal models. By choosing consensus sequences for therapeutically relevant gene families, novel targets can be identified that can be used in screening of drug candidates.

Thus, in a preferred embodiment, the present invention provides methods for isolating new members of gene families comprising introducing targeting polynucleotides comprising consensus homology clamps and at least one purification tag, preferably biotin, to a mix of nucleic acid, such as a plasmid cDNA library or a cell, and then utilizing the purification tag to isolate the gene(s). The exact methods will depend on the purification tag; a preferred method utilizes the attachment of the binding ligand for the tag to a bead, which is then used to pull out the sequence. Alternatively anti-RecA antibodies could be used to capture RecA-coated targeting polynucleotides. The genes are then cloned, sequenced, and reassembled if necessary, as is well known in the art.

Creation of Libraries of Variant Targets

In addition, the present invention allows for the introduction of insertions, deletions or substitutions in these cloned target sequences, to create libraries of variant targets that can subsequently be screened to identify useful variants.

Thus, in a preferred embodiment, the methods of the invention are used to generate pools or libraries of variant target nucleic acid sequences, and cellular libraries containing the variant libraries. This is distinct from the “gene shuffling” techniques of the literature (see Stemmer et al., 1994, Nature 370:389) which attempt to rapidly “evolve” genes by making multiple random changes simultaneously. In the present invention, this end is accomplished by using at least one cycle, and preferably reiterative cycles, of enhanced homologous recombination with targeting polynucleotides containing random mismatches. By using a library of targeting polynucleotides comprising a plurality of random mutations, and repeating the homologous recombination steps as many times as needed, a rapid “gene evolution” can occur, wherein the new genes may contain large numbers of mutations.

Thus, in this embodiment, a plurality of targeting polynucleotides is used. The targeting polynucleotides each have at least one homology clamp that substantially corresponds to or is substantially complementary to the target sequence. Generally, the targeting polynucleotides are generated in pairs; that is, pairs of two single stranded targeting polynucleotides that are substantially complementary to each other are made (i.e. a Watson strand and a Crick strand). However, as will be appreciated by those in the art, less than a one to one ratio of Watson to Crick strands may be used; for example, an excess of one of the single stranded target polynucleotides (i.e. Watson) may be used. Preferably, sufficient numbers of each of Watson and Crick strands are used to allow the majority of the targeting polynucleotides to form double D-loops, which are preferred over single D-loops as outlined above. In addition, the pairs need not have perfect complementarity; for example, an excess of one of the single stranded target polynucleotides (i.e. Watson), which may or may not contain mismatches, may be paired to a large number of variant Crick strands, etc. Due to the random nature of the pairing, one or both of any particular pair of single-stranded targeting polynucleotides may not contain any mismatches. However, generally, at least one of the strands will contain at least one mismatch.

The plurality of pairs preferably comprise a pool or library of mismatches. The size of the library will depend on a number of factors, including the number of residues to be mutagenized, the susceptibility of the protein to mutation, etc., as will be appreciated by those in the art. Generally, a library in this instance preferably comprises at least 10% different mismatches over the length of the targeting polynucleotides, with at least 30% mismatches being preferred and at least 40% being particularly preferred, although as will be appreciated by those in the art, lower (1, 2, 5%, etc.) or higher amounts of mismatches being both possible and desirable in some instances. That is, the plurality of pairs comprise a pool of random and preferably degenerate mismatches over some regions or all of the entire targeting sequence. As outlined herein, “mismatches” include substitutions, insertions and deletions, with the former being preferred. Thus, for example, a pool of degenerate variant targeting polynucleotides covering some, or preferably all, possible mismatches over some region are generated, as outlined above, using techniques well known in the art. Preferably, but not required, the variant targeting polynucleotides each comprise only one or a few mismatches (less than 10), to allow complete multiple randomization. That is, by repeating the homologous recombination steps any number of times, as is more fully outlined below, the mismatches from a plurality of targeting polynucleotides can be incorporated into a single target sequence.

The mismatches can be either non-random (i.e. targeted) or random, including biased randomness. That is, in some instances specific changes are desirable, and thus the sequence of the targeting polynucleotides are specifically chosen. In a preferred embodiment, the mismatches are random. The targeting polynucleotides can be chemically synthesized, and thus may incorporate any nucleotide at any position. The synthetic process can be designed to generate randomized nucleic acids, to allow the formation of all or most of the possible combinations over the length of the nucleic acid, thus forming a library of randomized targeting polynucleotides. Preferred methods maximize library size and diversity.

It is important to understand that in any library system encoded by oligonucleotide synthesis one cannot have complete control over the codons that will eventually be incorporated into the peptide structure. This is especially true in the case of codons encoding stop signals (TAA, TGA, TAG). In a synthesis with NNN as the random region, there is a 3/64, or 4.69%, chance that the codon will be a stop codon. To alleviate this, random residues are encoded as NNK, where K=T or G. This allows for encoding of all potential amino acids (changing their relative representation slightly), but importantly preventing the encoding of two stop residues TAA and TGA.

In one embodiment, the mismatches are fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

As will be appreciated by those in the art, the introduction of a pool of variant targeting polynucleotides (in combination with recombinase) to a target sequence, either in vitro to an extrachromosomal sequence or in vivo to a chromosomal or extrachromosomal sequence, can result in a large number of homologous recombination reactions occurring over time. That is, any number of homologous recombination reactions can occur on a single target sequence, to generate a wide variety of single and multiple mismatches within a single target sequence, and a library of such variant target sequences, most of which will contain mismatches and be different from other members of the library. This thus works to generate a library of mismatches.

In a preferred embodiment, the variant targeting polynucleotides are made to a particular region or domain of a sequence (i.e. a nucleotide sequence that encodes a particular protein domain). For example, it may be desirable to generate a library of all possible variants of a binding domain of a protein, without affecting a different biologically functional domain, etc. Thus, the methods of the present invention find particular use in generating a large number of different variants within a particular region of a sequence, similar to cassette mutagenesis but not limited by sequence length. This is sometimes referred to herein as “domain specific gene evolution”. In addition, two or more regions may also be altered simultaneously using these techniques; thus “single domain” and “multi-domain” shuffling can be performed. Suitable domains include, but are not limited to, kinase domains, nucleotide-binding sites, DNA binding sites, signaling domains, receptor binding domains, transcriptional activating regions, promoters, origins, leader sequences, terminators, localization signal domains, and, in immunoglobulin genes, the complementarity determining regions (CDR), Fc, V_{H} and V_{L}.

In a preferred embodiment, the variant targeting polynucleotides are made to the entire target sequence. In this way, a large number of single and multiple mismatches may be made in an entire sequence.

Thus, this embodiment proceeds as follows. A pool of targeting polynucleotides is made, each containing one or more mismatches. The targeting polynucleotides are coated with recombinase as generally described herein, and introduced to the target sequence as outlined herein. Upon binding of the targeting polynucleotides to form D-loops, homologous recombination can occur, producing altered target sequences. These altered target sequences can then be introduced into cells, if the shuffling was done in vitro, to produce target protein which can then be tested for biological activity, based on the identification of the target sequence. Depending on the results, the altered target sequence can be used as the starting target sequence in reiterative rounds of homologous recombination, generally using the same library. Preferred embodiments utilize at least two rounds of homologous recombination, with at least 5 rounds being preferred and at least 10 rounds being particularly preferred. Again, the number of reiterative rounds that are performed will depend on the desired end-point, the resistance or susceptibility of the protein to mutation, the number of mismatches in each targeting polynucleotide, etc.

Mutagenesis in vitro by Recombination

In addition to cloning target sequences such as genes or other nucleic acids or polynucleotides, the present invention also provides for high-throughput creation of variant target genes followed by phenotypic screening, as outlined below. That is, the present invention allows for the introduction of alterations in the target nucleic acid, in a high-throughput manner, generally using robotic systems. The resulting variants can be screened, again using high-throughput phenotypic screens, to identify useful variants. Thus, the fact that heterologies are tolerated in targeting polynucleotides allows for two things: first, the use of a heterologous consensus homology clamp that may target consensus functional domains of multiple genes, rather than a single gene, resulting in a variety of genotypes and phenotypes, and secondly, the introduction of alterations to the target sequence including insertion of heterologous DNA into the gene. Thus typically, a targeting polynucleotide (or complementary polynucleotide pair) has a portion or region having a sequence that is not present in the preselected endogenous targeted sequence(s) (i.e., a nonhomologous portion or mismatch) which may be as small as a single mismatched nucleotide, several mismatches, or may span up to about several kilobases or more of nonhomologous sequence.

Without binding to a particular theory, it is believed that the addition of recombinases to a targeting polynucleotide enhances the efficiency of homologous recombination between homologous, nonisogenic sequences (e.g., between an exon 2 sequence of an albumin gene of a Balb/c mouse and a homologous albumin gene exon 2 sequence of a C57/BL6 mouse), as well as between isogenic sequences.

The formation of heteroduplex joints is not a stringent process; genetic evidence supports the view that the classical phenomena of meiotic gene conversion and aberrant meiotic segregation results in part from the inclusion of mismatched base pairs in heteroduplex joints, and the subsequent correction of some of these mismatched base pairs before replication. Observations on RecA protein have provided information on parameters that affect the discrimination of relatedness from perfect or near-perfect homology and that affect the inclusion of mismatched base pairs in heteroduplex joints. The ability of RecA protein to drive strand exchange past all single base-pair mismatches and to form extensively mismatched joints in super-helical DNA reflects its role in recombination and gene conversion. This error-prone process may also be related to its role in mutagenesis. RecA-mediated pairing reactions involving DNA of FX174 and G4, which are about 70 percent homologous, have yielded homologous recombinants (Cunningham et al. (1981) Cell 24: 213), although RecA preferentially forms homologous joints between highly homologous sequences, and is implicated as mediating a homology search process between an invading DNA strand and a recipient DNA strand, producing relatively stable heteroduplexes at regions of high homology.

Accordingly, it is the fact that recombinases can drive the homologous recombination reaction between strands that are significantly, but not perfectly, homologous, which allows gene conversion and the modification of target sequences. Thus, targeting polynucleotides may be used to introduce nucleotide substitutions, insertions and deletions into an endogenous nucleic acid sequence, and thus the corresponding amino acid substitutions, insertions and deletions in proteins expressed from the endogenous nucleic acid sequence. By “endogenous” in this context herein is meant the naturally occurring sequence, i.e. sequences or substances originating from within a cell or organism. Similarly, “exogenous” refers to sequences or substances originating outside the cell or organism.

Mutagenesis in vivo by Recombination

In addition to cloning genes or modifying genes in vitro, this process can be used to modify, replace, remove or insert genes into cells or organisms in vivo. After the targeting polynucleotides are coated with recombinase, above, instead of using them to isolate or mutate genes in libraries, they are added to or inserted into cells. The targeting polynucleotides can be modified with cell-uptake components, chemical substituents, or the separation moieties outlined herein, etc.

In one embodiment, for example when the targeting polynucleotides are used to make alterations in a target sequence within cells, at least one of the targeting polynucleotides comprises at least one cell-uptake component. As used herein, the term “cell-uptake component” refers to an agent which, when bound, either directly or indirectly, to a targeting polynucleotide, enhances the intracellular uptake of the targeting polynucleotide into at least one cell type (e.g., hepatocytes). A targeting polynucleotide of the invention may optionally be conjugated, typically by covalent or preferably noncovalent binding, to a cell-uptake component. Various methods have been described in the art for targeting DNA to specific cell types. A targeting polynucleotide of the invention can be conjugated to essentially any of several cell-uptake components known in the art. For targeting to hepatocytes, a targeting polynucleotide can be conjugated to an asialoorosomucoid (ASOR)-poly-L-lysine conjugate by methods described in the art and incorporated herein by reference (Wu GY and Wu CH (1987) J. Biol. Chem. 262:4429; Wu GY and Wu CH (1988) Biochemistry 27:887; Wu GY and Wu CH (1988) J. Biol. Chem. 263: 14621; Wu GY and Wu CH (1992) J. Biol. Chem. 267: 12436; Wu et al. (1991) J. Biol. Chem. 266: 14338; and Wilson et al. (1992) J. Biol. Chem. 267: 963, WO92/06180; WO92/05250; and WO91/17761, which are incorporated herein by reference).

In addition to cellular uptake components, at least one of the targeting polynucleotides may include chemical substituents. Exogenous targeting polynucleotides that have been modified with appended chemical substituents may be introduced along with recombinase (e.g., RecA) into a metabolically active target cell to homologously pair with a predetermined endogenous DNA target sequence in the cell. In a preferred embodiment, the exogenous targeting polynucleotides are derivatized, and additional chemical substituents are attached, either during or after polynucleotide synthesis, and are thus localized to a specific endogenous target sequence where they produce an alteration or chemical modification to a local DNA sequence. Preferred attached chemical substituents include, but are not limited to: cross-linking agents (see Podyminogin et al., Biochem. 34:13098 (1995) and 35:7267 (1996), both of which are hereby incorporated by reference), nucleic acid cleavage agents, metal chelates (e.g., iron/EDTA chelate for iron catalyzed cleavage), topoisomerases, endonucleases, exonucleases, ligases, phosphodiesterases, photodynamic porphyrins, chemotherapeutic drugs (e.g., adriamycin, doxirubicin), intercalating agents, labels, base-modification agents, agents which normally bind to nucleic acids such as labels, etc. (see for example Afonina et al., PNAS USA 93:3199 (1996), incorporated herein by reference) immunoglobulin chains, and oligonucleotides. Iron/EDTA chelates are particularly preferred chemical substituents where local cleavage of a DNA sequence is desired (Hertzberg et al. (1982) J. Am. Chem. Soc. 104: 313; Hertzberg and Dervan (1984) Biochemistry 23: 3934; Taylor et al. (1984) Tetrahedron 40: 457; Dervan, PB ( 1986) Science 232: 464, which are incorporated herein by reference). Preferred groups include groups that prevent hybridization of the complementary single stranded nucleic acids to each other but not to unmodified nucleic acids (Kutryavin et al., Biochem. 35:11170 (1996) and Woo et al., Nucleic Acid. Res. 24(13):2470 (1996), both of which are incorporated by reference) and 2′-O methyl groups (Cole-Strauss et al., Science 273:1386 (1996); Yoon et al., PNAS 93:2071 (1996)). Additional preferred chemical substituents include labeling moieties, including fluorescent labels. Preferred attachment chemistries include: direct linkage, e.g., via an appended reactive amino group (Corey and Schultz (1988) Science 238:1401, which is incorporated herein by reference) and other direct linkage chemistries, although streptavidin/biotin and digoxigenin/antidigoxigenin antibody linkage methods may also be used. Methods for linking chemical substituents are provided in U.S. Pat. Nos. 5,135,720, 5,093,245, and 5,055,556, which are incorporated herein by reference. Other linkage chemistries may be used at the discretion of the practitioner.

Accordingly, in a preferred embodiment, the methods and compositions of the invention are used for inactivation of a gene. That is, exogenous targeting polynucleotides can be used to inactivate, decrease or alter the biological activity of one or more genes in a cell (or transgenic nonhuman animal or plant). This finds particular use in the generation of animal models of disease states, or in the elucidation of gene function and activity, similar to “knock out” experiments. Alternatively, the biological activity of the wild-type gene may be either decreased, or the wild-type activity altered to mimic disease states. This includes genetic manipulation of non-coding gene sequences that affect the transcription of genes, including, promoters, repressors, enhancers and transcriptional activating sequences.

Thus in a preferred embodiment, homologous recombination of the targeting polynucleotide and endogenous target sequence will result in amino acid substitutions, insertions or deletions in the endogenous target sequences, potentially both within the target sequence and outside of it, for example as a result of the incorporation of PCR tags. This will generally result in modulated or altered gene function of the endogenous gene, including a decrease or elimination of function as well as an enhancement of function.

Nonhomologous portions are used to make insertions, deletions, and/or replacements in a predetermined endogenous targeted DNA sequence, and/or to make single or multiple nucleotide substitutions in a predetermined endogenous target DNA sequence so that the resultant recombined sequence (i.e., a targeted recombinant endogenous sequence) incorporates some or all of the sequence information of the nonhomologous portion of the targeting polynucleotide(s). Thus, the nonhomologous regions are used to make variant sequences, i.e. targeted sequence modifications. In this way, site directed modifications may be done in a variety of systems for a variety of purposes.

The endogenous target sequence may be disrupted in a variety of ways. The term “disrupt” as used herein comprises a change in the coding or non-coding sequence of an endogenous nucleic acid. In one preferred embodiment, a disrupted gene will no longer produce a functional gene product. In another preferred embodiment, a disrupted gene produces a variant gene product. Generally, disruption may occur by either the substitution, insertion, deletion or frame shifting of nucleotides.

In one embodiment, amino acid substitutions are made. This can be the result of either the incorporation of a non-naturally occurring sequence into a target, or of more specific changes to a particular sequence outside of the sequence.

In one embodiment, the endogenous sequence is disrupted by an insertion sequence. The term “insertion sequence” as used herein means one or more nucleotides which are inserted into an endogenous gene to disrupt it. In general, insertion sequences can be as short as 1 nucleotide or as long as a gene, as outlined herein. For non-gene insertion sequences, the sequences are at least 1 nucleotide, with from about 1 to about 50 nucleotides being preferred, and from about 10 to 25 nucleotides being particularly preferred. An insertion sequence may comprise a polylinker sequence, with from about 1 to about 50 nucleotides being preferred, and from about 10 to 25 nucleotides being particularly preferred. Insertion sequence may be a PCR tag used for identification of the first gene.

In a preferred embodiment, an insertion sequence comprises a gene that not only disrupts the endogenous gene, thus preventing its expression, but also can result in the expression of a new gene product. Thus, in a preferred embodiment, the disruption of an endogenous gene by an insertion sequence gene is done in such a manner to allow the transcription and translation of the insertion gene. An insertion sequence that encodes a gene may range from about 50 bp to 5000 bp of cDNA or about 5000 bp to 50000 bp of genomic DNA. As will be appreciated by those in the art, this can be done in a variety of ways. In a preferred embodiment, the insertion gene is targeted to the endogenous gene in such a manner as to utilize endogenous regulatory sequences, including promoters, enhancers or a regulatory sequence. In an alternate embodiment, the insertion sequence gene includes its own regulatory sequences, such as a promoter, enhancer or other regulatory sequence etc.

Particularly preferred insertion sequence genes include, but are not limited to, genes which encode selection or reporter proteins. In addition, the insertion sequence genes may be modified or variant genes.

The term “deletion” as used herein comprises removal of a portion of the nucleic acid sequence of an endogenous gene. Deletions range from about 1 to about 100 nucleotides, with from about 1 to 50 nucleotides being preferred and from about 1 to about 25 nucleotides being particularly preferred, although in some cases deletions may be much larger, and may effectively comprise the removal of the entire consensus functional domain, the entire endogenous gene and/or its regulatory sequences. Deletions may occur in combination with substitutions or modifications to arrive at a final modified endogenous gene.

In a preferred embodiment, endogenous genes may be disrupted simultaneously by an insertion and a deletion. For example, some or all of an endogenous gene, with or without its regulatory sequences, may be removed and replaced with an insertion sequence gene. Thus, for example, all but the regulatory sequences of an endogenous gene may be removed, and replaced with an insertion sequence gene, which is now under the control of the endogenous gene's regulatory elements.

In addition, when the targeting polynucleotides are used to generate insertions or deletions in an endogenous nucleic acid sequence, as is described herein, the use of two complementary single-stranded targeting polynucleotides allows the use of internal homology clamps as depicted in the figures of PCT US98/05223. The use of internal homology clamps allows the formation of stable deproteinized cssDNA:targeting polynucleotide target hybrids with homologous DNA sequences containing either relatively small or large insertions and deletions within a homologous DNA target. Without being bound by theory, it appears that these targeting polynucleotide:target hybrids, with heterologous inserts in the cssDNA targeting polynucleotide, are stabilized by the re-annealing of cssDNA targeting polynucleotides to each other within the double-D-loop hybrid, forming a novel DNA structure with an internal homology clamp. Similarly stable double-D-loop hybrids formed at internal sites with heterologous inserts in the linear DNA targets (with respect to the cssDNA targeting polynucleotide) are equally stable. Because cssDNA targeting polynucleotides are kinetically trapped within the duplex target, the multi-stranded DNA intermediates of homologous DNA pairing are stabilized and strand exchange is facilitated. In addition, internal homology clamps may be used for cloning, as well.

In a preferred embodiment, the length of the internal homology clamp (i.e. the length of the insertion or deletion) is from about 1 to 50% of the total length of the targeting polynucleotide, with from about 1 to about 20% being preferred and from about 1 to about 10% being especially preferred, although in some cases the length of the deletion or insertion may be significantly larger. As for the consensus homology clamps, the complementarity within the internal homology clamp need not be perfect.

Recombinase protein(s) (prokaryotic, eukaryotic or endogenous to the target cell) may be exogenously induced or administered to a target cell or nucleic acid library simultaneously or contemporaneously (i.e., within about a few hours) with the targeting polynucleotide(s). Such administration is typically done by micro-injection, although electroporation, lipofection, and other transfection methods known in the art may also be used. Alternatively, recombinase-proteins may be produced in vivo. For example, they may be produced from a homologous or heterologous expression cassette in a transfected cell or targeted cell, such as a transgenic totipotent cell (e.g. a fertilized zygote) or an embryonic stem cell (e.g., a murine ES cell such as AB-1) used to generate a transgenic non-human animal line or a somatic cell or a pluripotent hematopoietic stem cell for reconstituting all or part of a particular stem cell population (e.g. hematopoietic) of an individual. Conveniently, a heterologous expression cassette includes a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible drug inducible, or other modulatable promoter construct so that expression of at least one species of recombinase protein from the cassette can by modulated for transiently producing recombinase(s) in vivo simultaneous or contemporaneous with introduction of a targeting polynucleotide into the cell. When a hormone-inducible promoter-enhancer combination is used, the cell must have the required hormone receptor present, either naturally or as a consequence of expression a co-transfected expression vector encoding such receptor. Alternatively, the recombinase may be endogenous and produced in high levels. In this embodiment, preferably in eukaryotic target cells such as tumor cells, the target cells produce an elevated level of recombinase. In other embodiments the level of recombinase may be induced by DNA damaging agents, such as mitomycin C, UV or -irradiation. Alternatively, recombinase levels may be elevated by transfection of a plasmid encoding the recombinase gene into the cell.

Alternatively, a cell-uptake component may be formed by incubating the targeting polynucleotide with at least one lipid species and at least one protein species to form protein-lipid-polynucleotide complexes consisting essentially of the targeting polynucleotide and the lipid-protein cell-uptake component. Lipid vesicles made according to Felgner (WO91/17424, incorporated herein by reference) and/or cationic lipidization (WO91/16024, incorporated herein by reference) or other forms for polynucleotide administration (EP 465,529, incorporated herein by reference) may also be employed as cell-uptake components. Nucleases may also be used.

In addition to cell-uptake components, targeting components such as nuclear localization signals may be used, as is known in the art. See for example Kido et al., Exper. Cell Res. 198:107-114 (1992), hereby expressly incorporated by reference.

Typically, a targeting polynucleotide of the invention is coated with at least one recombinase and is conjugated to a cell-uptake component, and the resulting cell targeting complex is contacted with a target cell under uptake conditions (e.g., physiological conditions) so that the targeting polynucleotide and the recombinase(s) are internalized in the target cell. A targeting polynucleotide may be contacted simultaneously or sequentially with a cell-uptake component and also with a recombinase; preferably the targeting polynucleotide is contacted first with a recombinase, or with a mixture comprising both a cell-uptake component and a recombinase under conditions whereby, on average, at least about one molecule of recombinase is noncovalently attached per targeting polynucleotide molecule and at least about one cell-uptake component also is noncovalently attached. Most preferably, coating of both recombinase and cell-uptake component saturates essentially all of the available binding sites on the targeting polynucleotide. A targeting polynucleotide may be preferentially coated with a cell-uptake component so that the resultant targeting complex comprises, on a molar basis, more cell-uptake component than recombinase(s). Alternatively, a targeting polynucleotide may be preferentially coated with recombinase(s) so that the resultant targeting complex comprises, on a molar basis, more recombinase(s) than cell-uptake component.

Cell-uptake components are included with recombinase-coated targeting polynucleotides of the invention to enhance the uptake of the recombinase-coated targeting polynucleotide(s) into cells, particularly for in vivo gene targeting applications, such as gene therapy to treat genetic diseases, including neoplasia, and targeted homologous recombination to treat viral infections wherein a viral sequence (e.g., an integrated hepatitis B virus (HBV) genome or genome fragment) may be targeted by homologous sequence targeting and inactivated. Alternatively, a targeting polynucleotide may be coated with the cell-uptake component and targeted to cells with a contemporaneous or simultaneous administration of a recombinase (e.g., liposomes or immunoliposomes containing a recombinase, a viral-based vector encoding and expressing a recombinase).

When using microinjection procedures it may be preferable to use a transfection technique with linearized sequences containing only modified target gene sequence and without vector or selectable sequences. The modified gene site is such that a homologous recombinant between the exogenous targeting polynucleotide and the endogenous DNA target sequence can be identified by using carefully chosen primers and PCR, followed by analysis to detect if PCR products specific to the desired targeted event are present (Erlich et al., (1991) Science 252: 1643, which is incorporated herein by reference). Several studies have already used PCR to successfully identify and then clone the desired transfected cell lines (Zimmer and Gruss, (1989) Nature 338: 150; Mouellic et al., (1990) Proc. Natl. Acad. Sci. USA 87: 4712; Shesely et al., (1991) Proc. Natl. Acad. Sci. USA 88: 4294, which are incorporated herein by reference). This approach is very effective when the number of cells receiving exogenous targeting polynucleotide(s) is high (i.e., with microinjection, or with liposomes) and the treated cell populations are allowed to expand to cell groups of approximately 1×104 cells (Capecchi, (1989) Science 244: 1288). When the target gene is not on a sex chromosome, or the cells are derived from a female, both alleles of a gene can be targeted by sequential inactivation (Mortensen et al., (1991) Proc. Natl. Acad. Sci. USA 88: 7036). Alternatively, animals heterologous for the target gene can be bred to homologously as is known in the art.

In some embodiments, for example when phenotypic screens are to be done, the targeting polynucleotides are introduced into target cells, as defined herein. In a preferred embodiment, the target sequence is a chromosomal sequence. In this embodiment, the recombinase with the targeting polynucleotides are introduced into the target cell, preferably eukaryotic target cells. In this embodiment, it may be desirable to bind (generally non-covalently) a nuclear localization signal to the targeting polynucleotides to facilitate localization of the complexes in the nucleus. See for example Kido et al., Exper. Cell Res. 198:107-114 (1992), hereby expressly incorporated by reference.

Similarly, in some embodiments, for some screens, preferred eukaryotic cells are embryonic stem cells (ES cells) and fertilized zygotes are preferred. In a preferred embodiment, embryonic stem cells are used. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 21-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germline transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

In a preferred embodiment, non-human zygotes are used, for example to make transgenic animals, using techniques known in the art (see U.S. Pat. No. 4,873,191). Preferred zygotes include, but are not limited to, animal zygotes, including fish, avian and mammalian zygotes. Suitable fish zygotes include, but are not limited to, those from species of salmon, trout, tuna, carp, flounder, halibut, swordfish, cod, tilapia and zebra fish. Suitable bird zygotes include, but are not limited to, those of chickens, ducks, quail, pheasant, turkeys, and other jungle fowl and game birds. Suitable mammalian zygotes include, but are not limited to, cells from horses, cows, buffalo, deer, sheep, rabbits, rodents such as mice, rats, hamsters and guinea pigs, goats, pigs, primates, and marine mammals including dolphins and whales. See Hogan et al., Manipulating the Mouse Embryo (A Laboratory Manual), 2nd Ed. Cold Spring Harbor Press, 1994, incorporated by reference.

For screening, the vectors containing the compositions of the invention can be transferred into the host cell by well-known methods, depending on the type of cellular host. For example, micro-injection is commonly utilized for target cells, although calcium phosphate treatment, electroporation, lipofection, biolistics or viral-based transfection also may be used. Other methods used to transform mammalian cells include the use of Polybrene, protoplast fusion, and others (see, generally, Sambrook et al. Molecular Cloning: A Laboratory Manual, 2d ed., 1989, Cold Spring Harbor Laboratory Press, Cold Spring Harbor, N.Y., which is incorporated herein by reference). Direct injection of DNA and/or recombinase-coated targeting polynucleotides into target cells, such as skeletal or muscle cells also may be used (Wolff et al. (1990) Science 247: 1465, which is incorporated herein by reference).

DNA Arrays

It should be noted that the entire or any part of the gene cloning reactions can occur in solution, in cell extracts, in cells, in organisms, or on solid supports or in arrays. Any part of the gene cloning reaction can occur on microplates, microarrays, or any other solid supports such as beads, glass, silica chips, filters, fibers including optical fibers, metallic or plastic supports, ceramics, other sensors, etc.

The cloning reactions outlined herein can be done on a solid support. Thus, as is known in the art, there are a wide variety of different types of nucleic acid arrays on solid supports (frequently referred to in the art as “gene chips”, “biochips”, “probe arrays”, microbead flow cells etc.). These comprise nucleic acids attached to a solid support in a variety of ways, including covalent and non-covalent attachments. By adding recombinases to gene chips, the targeting polynucleotides on the surface become a first targeting polynucleotide as outlined herein. Optionally, one or more of the second targeting polynucleotides may be added to the reaction mixture; that is, this can be done in a highly parallel way by including the substantially complementary strands to the targeting polynucleotides on the surface. However, as outlined herein, single D-loops are stable as well, so this may not be required. Then, by adding a cDNA library to the chip, as is done above for the single reactions, the target sequences hybridize to the targeting polynucleotides. Washing the unhybridized nucleic acids away, followed by elution, amplification if required and sequencing of the targets allows the simultaneous cloning of a number of genes simultaneously. In this embodiment, a separation moiety may not be required.

Automation of EHR Technology

Automation of EHR technology enables high-throughput gene cloning, high throughput phenotypic screening and identification, and biovalidation of drug targets simultaneously from multiple cell types, tissues and organisms. Preferably, the automated methods and compositions of the invention comprise a robotic system. The systems outlined herein are generally directed to the use of 96- or 384-well microtiter plates, but as will be appreciated by those in the art, any number of different plates or configurations may be used. In addition, any or all of the steps outlined herein may be automated; thus, for example, the systems may be completely or partially automated.

Referring to FIG. 1, a preferred embodiment of the present invention has eight modules to form a novel Integrated Genomic Handling System™. Module 1 is directed to automated design and synthesis of the targeting polynucleotides. Module 2 is directed to automated gene cloning using the targeting polynucleotides from Module 1 and the novel enhanced homologous recombination methods (EHR) of the present invention. Module 3 is directed to automated transformation and amplification of cloned genes from Module 2. Module 4 is directed to automated verification and culturing of transformed cells from Module 3. Module 5 is directed to automated isolation and purification of cloned DNA from the cells of Module 4. Module 6 is directed to automated analysis and identification of the isolated cloned DNA. Module 7 is directed to automated sequencing of the isolated clone. Module eight is directed to database(s) used to store and retrieve information. As will be appreciated by the skilled artisan, more or less than eight modules may be used and the number of modules discussed herein is based upon the best mode of practicing the present invention at the time of filing this application.

As will be appreciated by those in the art, an automated system can include a wide variety of components, including, but not limited to, liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and computer systems.

The robotic systems include automated liquid and particle handling enabling high throughput pipetting to perform all the steps in the process of gene targeting and recombination applications. This includes liquid and particle manipulations such as aspiration, dispensing, mixing, diluting, washing, accurate volumetric transfers; retrieving and discarding of pipette tips; and repetitive pipetting of identical volumes for multiple deliveries from a single sample aspiration. These manipulations are cross-contamination-free liquid, particle, cell, and organism transfers. The instruments performs automated replication of microplate samples to filters, membranes, and/or daughter plates, high-density transfers, full-plate serial dilutions, and high capacity operation.

In a preferred embodiment, chemically derivatized particles, plates, filters, tubes, magnetic particles, or other solid phase matrix with specificity to the ligand or recognition groups on the DNA targeting polynucleotide, recombinase protein or peptide are used to isolate the targeted DNA hybrids. The binding surfaces of microplates, tubes, filters or beads, or any solid phase matrices including non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrices are useful in this invention to capture the targeted DNA hybrids.

In a preferred embodiment, platforms for multi-well plates, deep-well plates, square well plates, reagent troughs, test tubes, mini tubes, microfuge tubes, cryovials, filters, micro array chips, optic fibers, beads, agarose and acrylamide gels, and other solid-phase matrices or platforms are accommodated on an upgradeable modular deck. This modular deck includes multi-position work surfaces for placing source and output samples, reagents, sample and reagent dilution, assay plates, sample and reagent reservoirs, pipette tips, and an active tip-washing station.

In a preferred embodiment, an integrated thermal cycler and thermal regulators are used for stabilizing the temperature of heat exchangers such as controlled blocks or platforms to provide accurate temperature control of incubating samples from 4° C. to 100° C.

In a preferred embodiment, interchangeable machine-heads (single or multi-channel) with single or multiple magnetic probes, affinity probes, replicators or pipetters, robotically manipulate the liquid, particles, cells, and organisms. Multi-well or multi-tube magnetic separators and filtration stations manipulate liquid, particles, cells, and organisms in single or multiple sample formats.

The flexible hardware and software allow instrument adaptability for multiple applications. The software program modules allow creation, modification, and running of methods. The system's diagnostic modules allow setup, instrument alignment, and motor operations. The customized tools, labware, and liquid and particle transfer patterns allow different applications to be programmed and performed. The database allows method and parameter storage. Robotic and computer interfaces allow communication between instruments.

In a preferred embodiment, the robotic workstation includes one or more heating or cooling components. Depending on the reactions and reagents, either cooling or heating may be required, which can be done using any number of known heating and cooling systems, including Peltier systems.

In a preferred embodiment, the robotic apparatus includes a central processing unit (CPU) that communicates with a memory and a set of input/output devices (e.g., keyboard, mouse, monitor, printer, etc.) through a bus. The general interaction between a central processing unit, a memory, input/output devices, and a bus is known in the art. A variety of different procedures, depending on the experiments to be run, are stored in the CPU memory.

Module 1 Library Validation and Targeting Polynucleotide Synthesis and Purification

Referring to FIG. 10, a target nucleic acid sequence (for example an EST, gene sequence, or consensus sequence) is input into a computer system, and Module 1 designs and automates the synthesis and purification of the targeting polynucleotide(s) (also referred to herein as probes). PCR primers are designed for the target sequence of interest (for example an EST or gene sequence), which are used in a PCR reaction to amplify fragments of an expected size from a cDNA library, a genomic DNA library, or other library of DNA. Preferably, primers and PCR amplification is used to verify that the target sequence is present in a library. After the presence of the target is verified, one set of primers is used to synthesize the targeting polynucleotide for the capture of the targeted gene using PCR technology. The probe or targeting polynucleotide is purified to remove free nucleotides and is quantitated and diluted to a standard working concentration.

In a preferred embodiment, a robotic station for Module 1 includes a micro-processor controlled liquid handler with a multi-channel pipettor head, equipped with a robotic arm for moving plates between deck positions (examples include, but are not limited to, Tecan Genesis, Beckman Coulter Biomek 2000, Beckman Coulter Biomek FX). The microprocessor runs a managing software program that coordinates the different components of Module 1, and coordinates Module 1 with the other Modules described herein. The liquid handler preferably includes an integrated thermal cycler block on the deck, or alternatively the thermal cycler can be integrated with the liquid handler by the means of a robotic arm (examples include, but are not limited to, MJ Research DNA Engine and DNA Tetrad thermal cyclers, MWG Primus thermal cycler, and any other thermal cycler with a motorized lid that can be controlled remotely). The thermal cycler is controlled by the managing software program. Module 1 also preferably includes a magnetic bead processing unit, or a filtration device (for purifying the PCR products), a plate sealer that seals the plates prior to insertion into the thermal cycler (examples include, but are not limited to the Marsh thermal sealer for microplates, Velocity 11 plate sealer, Wako plate sealer, and any other thermal sealer for microplates that can be integrated robotically and controlled remotely), a piercing device that allows the piercing of the seal while holding the plate in place, a barcode reader and applicator, a gel loading device that allows the liquid handler to load electrophoresis gels, and a plate reader capable of reading absorbance at 260 nm, and/or performing DNA fluorometric measurements in 96 or 384-well format (examples include, but are not limited to, Tecan SpectraFluor and SpectraFluor Plus, BMG FluoStar Galaxy, Perkin Elmer LS and Molecular Devices SPECTRAmax Gemini XS). The components are preferably integrated on the liquid handler deck, or alternatively are integrated into the system by a robotic arm. Referring to FIG. 10 the steps of a preferred embodiment of Module 1 follow:

1. To facilitate a high throughput, a user enters the sequence(s) of multiple target(s) into a target sequence database. The software then processes the entries of the sequence database to generate a target input file, which is sent to the primer design software. This software designs the primers, and outputs the results into a target primer file. The software then processes the target primer file, and submits an oligonucleotide order form to an oligonucleotide synthesizing facility, as will be understood within the art. Preferably, two sets of primers are designed, one set is nested inside the other. The nested set is called the targeting sequence primers, while the other set is called the verification primers.

2. When the oligonucleotides are available, the user sets up the deck of the liquid handler by placing the required reagents, primers and source for target DNA in the appropriate positions on the deck, as will be known in the art.

3. The liquid handler, controlled by the microprocessor and associated software, sets up PCR reactions in a multi-well plate, using the primers and the desired source for target DNA. Preferably, all four primer-combinations are tested. The locations within the plate and estimated sizes of PCR products are entered into the database.

4. The plate is then sealed by the plate sealer and moved into the thermal cycler with a motorized lid. The microprocessor issues the command to close the lid, and starts the cycling. The number of cycles used will be apparent to and at the discretion of the practitioner, or the software may be designed to select the number of cycles.

5. When the amplification is complete, the plate is removed out of the cycler by the robotic arm, the seal is pierced robotically, and an aliquot of the PCR products is loaded onto a gel using the gel loading device. The gel is electrophoresed, and inspected visually to verify that a single amplified product is present in each well of the gel. As will be appreciated by the skilled artisan, visual inspection may be done manually or using an optical reader connected to the microprocessor and robotic system of Module 1.

6. The liquid handler uses the appropriate PCR products as templates for setting up new PCR reactions to generate and separate the targeting polynucleotide as described in steps 2-5. In this PCR reaction the targeting polynucleotide is generated, by incorporating a separation moiety into the reaction (e.g. biotinylated nucleotides), as is known within the art. The locations and estimated sizes of probes are entered into the database.

7. Contaminants are removed from the targeting polynucleotide(s), preferably by magnetic bead based protocol, as is known within the art. The targeting polynucleotide(s) (i.e., the PCR products) are preferentially bound to magnetic particles allowing the removal of primers, primer-dimers and unincorporated nucleotides from the targeting polynucleotide(s). The process is preferably carried out in a 96-well plate format, utilizing the magnetic bead processing platform on the deck of the robot.

8. The purified targeting polynucleotide(s) are quantitated, either by absorbance of UV light, or by a fluorometric analysis, preferably using the plate reader, and the concentration is stored into the database.

9. The software calculates the required dilution to bring the separated targeting polynucleotide(s) to a fixed working concentration, preferably 1 μg/μl, and the liquid handler then dilutes the targeting polynucleotide(s) into a new plate. Information about targeting polynucleotide(s) concentration and location within the plate is entered into the database.

As will be appreciated by those in the art, the robotic system of the present invention preferably utilizes software to perform all the required steps, calculations and analysis done within Module 1. In addition to specific software that controls the liquid handler, a central control software program coordinates different components within Module 1 and between Module 1 and the other Modules described herein. For example, the central control software program can initiate the process for the liquid handler to move the plate into a thermal cycler block, close the lid of the block, start the cycling procedure on the thermal cycler, resume operation on the deck of the liquid handler, and, at the end of the cycling program, open the lid of the thermal cycler, and instruct the liquid handler to remove the plate and place it back on the deck. As will be appreciated by the skilled artisan, there may be many different software programs controlled by a supervising or managing program, however, the hierarchy of the software is not critical to present invention.

Module 2: Target Capture

Referring to FIG. 11, Module 2 is directed to automated gene cloning methods preferably utilizing enhanced homologous recombination techniques. Generally the steps include the denaturation of the targeting polynucleotide(s), coating the single-stranded targeting polynucleotide(s) with a recombinase (preferably RecA), targeting of cssDNA targeting polynucleotides to target DNA by formation of probe:target hybrids, and capture of the probe:target hybrids. As described above, targeting single stranded polynucleotides need not be perfectly complementary to each other. Preferably the single-stranded targeting polynucleotides are at least about 95% complementary to each other, but, as described above, can be as little as about 50% complementary.

In a preferred embodiment, a robotic station for Module 2 includes a micro-processor controlled liquid handler with a multi-channel pipettor head, ideally equipped with a robotic arm for moving plates between deck positions (examples include, but are not limited to, Tecan Genesis, Beckman Coulter Biomek 2000, Beckman Coulter Biomek FX). Preferably, the microprocessor of Module 2 is the same as for Module 1 and the other Modules described herein, although this is not required. The microprocessor runs a managing software program that coordinates the different components of Module 2, which is preferably the same managing software program or a subroutine thereof that coordinates the different components of Module 1 and the components of the other Modules described herein, although this is not required. Preferably, the liquid handler includes an integrated thermal cycler block on the deck, or alternatively a thermal cycler can be integrated with the liquid handler by a robotic arm (examples include, but are not limited to, MJ Research DNA Engine and DNA Tetrad thermal cyclers, MWG Primus thermal cycler, and any other thermal cycler with a motorized lid that can be controlled remotely). The thermal cycler is controlled by the managing software program. Preferably Module 2 also includes a magnetic bead processing unit, a plate sealer that seals the plates prior to insertion into the thermal cycler, a piercing device that allows the piercing of the seal while holding the plate in place, a barcode reader and applicator, and a shaker. The components can be integrated on the liquid handler deck, or they can be integrated into the system by a robotic arm.

Referring to FIG. 11 the steps of a preferred embodiment of Module 2 follow:

1. The user sets up the deck of the liquid handler by placing the different components (targeting polynucleotide(s), recombinase coating solution, deproteinization solution, PMSF, wash buffer etc.) in the liquid handler. For preparation of the coating solution, for each reaction 6 ml of the 5×coating buffer (50 mM Tris-acetate, pH 7.5, 250 mM sodium-acetate, 10 mM Mg-Acetate, and 5 mM DTT), 3.7 ml of 16.2 mM ATPgS (Boehringer Mannheim), and 0.7 ml of 1 mg/ml RecA (Promega) protein (total of 10.4 ml per reaction) are combined in a single tube which is placed in a 4° C. cooled position of a reagent rack on the robot deck. For preparation of the deproteinization solution, for each reaction, 0.6 ml of SDS solution (10 mg/ml) and 0.4 ml of Proteinase K (Boehringer Mannheim) are combined in a single 0.5 ml microfuge tube and placed in the reagent rack.

2. The liquid handler dispenses 5 ml of each targeting polynucleotide (50 ng) into wells in a microtiter PCR plate.

3. A robotic arm moves the plate to a plate sealer that seals the plate, and then into a thermal cycler block.

4. The thermal cycler heats the samples to 95° C. for 3 minutes, and then chills them to 4° C. for 5 minutes. As will be appreciated by those in the art, other types of denaturing may be done, for example chemical denaturants may be used. In addition, all subsequent steps may be done at room temperature.

5. A robotic arm removes the plate from the thermal cycler block, and returns it to the plate piercer, which pierces the seal.

6. The liquid handler transfers 3 ml from the content of the wells into a new plate. The liquid handler dispenses 10.4 ml of the coating mixture into each well, the mixture is mixed, preferably by pipetting up and down, and the plate is transferred to a thermally controlled position, where it is incubated at 37° C. for 15 minutes.

7. The liquid handler dispenses target DNA (5 mg in a volume of 5 ml) and 1.2 ml of 200 mM Mg-Acetate into the wells with the nucleoprotein filaments, the contents of the wells are mixed, preferably by pipetting up and down, and the mixture incubated further for 20 minutes.

8. The liquid handler dispenses 1 ml of 50-mg/ml salmon sperm competitor DNA into each well, the contents of the wells are mixed, preferably by pipetting up and down, and the mixture incubated further for 5 minutes.

9. The liquid handler dispenses 1 ml of the deproteinization solution into each well of the sample microplate and optionally mixes the samples by pipetting. The microplate is further incubated for 10 minutes at 37° C.

10. The liquid handler dispenses 1 ml of 0.1M phenylmethyl-sulfonyl fluoride (PMSF) protease inhibitor (Boehringer Mannheim) from the reagent rack.

11. The liquid handler dispenses an appropriate amount of streptavidin-coated magnetic beads into each well.

12. The plate is transferred to a shaker, and is shaken for 30 minutes to allow binding of the biotinylated DNA (probe:target hybrids) to the magnetic particles.

13. The plate is transferred to a magnetic position, and held above the magnet for enough time to allow the particles to settle.

14. The liquid handler aspirates the supernatant.

15. The plate is transferred to a non-magnetic position, or the magnets are disengaged in the current position, so that there is no magnetic field.

16. The liquid handler dispenses wash buffer (10 mM Tris-HCl pH 7.5, 2 M NaCl, and 1 mM EDTA) into the wells, and pipettes the solution up and down a few times to wash the particles.

17. Steps 13-16 are repeated for a total of 4 washes.

18. The plate is transferred to a thermally controlled position preheated to 85° C.

19. The liquid handler adds 8 ml of elution solution (low salt buffer). The mixture is incubated at 85° C. for 5 minutes, and is then transferred back to the magnetic position.

20. The particles are allowed to settle, and the supernatant is aspirated and transferred into a fresh microtiter plate.

In this preferred embodiment, the targeting polynucleotides are coated with recombinase prior to introduction to the target, although recombinase and targeting polynucleotides may be introduced separately or simultaneously to the target DNA. The conditions used to coat targeting polynucleotides with recombinases such as RecA protein and ATPgS have been described in commonly assigned U.S. Ser. No. 07/910,791, filed Jul. 9, 1992; U.S. Ser. No. 07/755,462, filed Sep. 4, 1991; and U.S. Ser. No. 07/520,321, filed May 7, 1990, and PCT US98/05223, each incorporated herein by reference. The procedures below are directed to the use of E. coli RecA, although as will be appreciated by those in the art, other recombinases may be used as well.

Targeting polynucleotides can be coated using GTPgS, mixes of ATPgS with rATP, rGTP and/or dATP, or dATP or rATP alone in the presence of a rATP generating system (Boehringer Mannheim). Various mixtures of GTPg S, ATPgS, ATP, ADP, dATP and/or rATP or other nucleosides may be used, particularly preferred are mixes of ATPgS and ATP or ATPgS and ADP.

RecA protein coating of targeting polynucleotides is typically carried out as described in U.S. Ser. No. 07/910,791, filed Jul. 9, 1992 and U.S. Ser. No. filed Sep. 4, 1991, and PCT US98/05223, which are incorporated herein by reference. Briefly, the targeting polynucleotide, whether double-stranded or single-stranded, is denatured by heating in an aqueous solution at 95 100° C. for five minutes, then placed in an ice bath for 20 seconds to about one minute followed by centrifugation at 0° C. for approximately 20 sec, before use. When denatured targeting polynucleotides are not placed in a freezer at −20° C. they are usually immediately added to standard RecA coating reaction buffer containing ATPgS, at room temperature, and to this is added the RecA protein. Alternatively, RecA protein may be included with the buffer components and ATPgS before the polynucleotides are added.

RecA coating of targeting polynucleotide(s) is initiated by incubating polynucleotide-RecA mixtures at 37° C. for 10-15 min. RecA protein concentration tested during reaction with polynucleotide varies depending upon polynucleotide size and the amount of added polynucleotide, and the ratio of RecA molecule:nucleotide preferably ranges between about 3:1 and 1:3. When single-stranded polynucleotides are RecA coated independently of their homologous polynucleotide strands, the concentrations of ATPgS and RecA can be reduced to one-half those used with double-stranded targeting polynucleotides (i.e., RecA and ATPgS concentration ratios are usually kept constant at a specific concentration of individual polynucleotide strand, depending on whether a single- or double-stranded polynucleotide is used).

RecA protein coating of targeting polynucleotides is normally carried out in a standard 1×RecA coating reaction buffer. 10×RecA reaction buffer (i.e., 10×AC buffer) consists of: 100 mM Tris acetate (pH 7.5 at 37 C.), 20 mM magnesium acetate, 500 mM sodium acetate, 10 mM DTT, and 50% glycerol). All of the targeting polynucleotides, whether double-stranded or single-stranded, typically are denatured before use by heating to 95-100° C. for five minutes, placed on ice for one minute, and subjected to centrifugation (10,000 rpm) at 0° C. for approximately 20 seconds (e.g., in a Tomy centrifuge). Denatured targeting polynucleotides usually are added immediately to room temperature RecA coating reaction buffer mixed with ATPgS and diluted with double-distilled H₂O as necessary.

A reaction mixture typically contains the following components: (i) 0.2-4.8 mM ATPgS; and (ii) between 1-100 ng/ml of targeting polynucleotide. To this mixture is added about 1-20 ml of RecA protein per 10-100 ml of reaction mixture, usually at about 2-10 mg/ml (purchased from Pharmacia or purified), which is rapidly added and mixed. The final reaction volume for RecA coating of targeting polynucleotide is usually in the range of about 10-500 ml. RecA coating of targeting polynucleotide is usually initiated by incubating targeting polynucleotide-RecA mixtures at 37° C. for about 10-15 min.

RecA protein concentration in coating reactions varies depending upon targeting polynucleotide size and the amount of added targeting polynucleotide. RecA protein concentrations are typically in the range of 5 to 50 mM. When single-stranded targeting polynucleotides are coated with RecA, independently of their complementary strands, the concentrations of ATPgS and RecA protein may optionally be reduced to about one-half of the concentrations used with double-stranded targeting polynucleotides of the same length: that is, the RecA protein and ATPgS concentration ratios are generally kept constant for a given concentration of individual polynucleotide strands.

The coating of targeting polynucleotides with RecA protein can be evaluated in a number of ways. First, protein binding to DNA can be examined using band-shift gel assays (McEntee et al., (1981) J. Biol. Chem. 256: 8835). Labeled polynucleotides can be coated with RecA protein in the presence of ATPgS and the products of the coating reactions may be separated by agarose gel electrophoresis. Following incubation of RecA protein with denatured duplex DNAs the RecA protein effectively coats single-stranded targeting polynucleotides derived from denaturing a duplex DNA. As the ratio of RecA protein monomers to nucleotides in the targeting polynucleotide increases from 0, 1:27, 1:2.7 to 3.7:1 for 121-mer and 0, 1:22, 1:2.2 to 4.5:1 for 159-mer, the targeting polynucleotides' electrophoretic mobility decreases, i.e., is retarded, due to RecA-binding to the targeting polynucleotide. Retardation of the coated polynucleotide's mobility reflects the saturation of targeting polynucleotide with RecA protein. An excess of RecA monomers to DNA nucleotides is required for efficient RecA coating of short targeting polynucleotides (Leahy et al., (1986) J. Biol. Chem. 261: 954).

A second method for evaluating protein binding to DNA is in the use of nitrocellulose filter binding assays (Leahy et al., (1986) J. Biol. Chem. 261:6954; Woodbury, et al., (1983) Biochemistry 22(20):4730-4737. The nitrocellulose filter binding method is particularly useful in determining the dissociation-rates for protein:DNA complexes using labeled DNA. In the filter binding assay, DNA:protein complexes are retained on a filter while free DNA passes through the filter. This assay method is more quantitative for dissociation-rate determinations because the separation of DNA:protein complexes from free targeting polynucleotide is very rapid.

In a preferred embodiment, the compositions find use in the cloning of target nucleic acids. In this embodiment, the EHR compositions are contacted with a nucleic acid composition such as a cDNA library, genomic DNA, or YAC, BAC or PAC libraries. In general, any composition of nucleic acid that serves as a source of target sequences can be used. In addition, the target can be genomic DNA, plasmid DNA, cDNA, or RNA, either in a library of replicative vectors or as a collection of non-replicating DNA fragments. In addition, any target cells outlined herein may be used to generate a cDNA library for use in the invention. Furthermore, while not preferred in some embodiments, the nucleic acid library may actually be a library of target cells.

Thus, in a preferred embodiment, the methods of the invention comprise contacting the compositions of the invention with a nucleic acid library to clone target sequences. The nucleic acid libraries may be made from any number of different target cells as is known in the art. By “target cells” herein is meant prokaryotic or eukaryotic cells. Suitable prokaryotic cells include, but are not limited to, bacteria such as E. coli, Bacillus species, and extremophile bacteria such as thermophiles, etc. Preferably, the prokaryotic target cells are recombination competent. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cotton, tomato, rice, potato, alfalfa, sunflower, etc.; and animal cells, including fish, avian and mammalian cells. Suitable fish cells include, but are not limited to, those from species of salmon, trout, tilapia, tuna, carp, flounder, halibut, swordfish, cod and zebra fish. Suitable avian cells include, but are not limited to, those of chicken, duck, quail, pheasant and turkey, and other jungle foul or game birds. Suitable mammalian cells include, but are not limited to, cells from horse, cow, buffalo, deer, sheep, rabbit, rodents such as mouse, rat, hamster and guinea pig, goat, pig, primates, marine mammals including dolphins and whales, as well as cell lines, such as human cell lines of any tissue or stem cell type, and stem cells, including pluripotent and non-pluripotent, and non-human zygotes. In some embodiments, preferred cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In a preferred embodiment, prokaryotic cells are used as the target. In one embodiment, the target sequence is contained within an extrachromosomal sequence. By “extrachromosomal sequence” herein is meant a sequence separate from the chromosomal or genomic sequences. Preferred extrachromosomal sequences include plasmids (particularly prokaryotic plasmids such as bacterial plasmids), p1 vectors, viral genomes (including retroviruses and adenoviruses and other viruses that can be used to put altered genes into eukaryotic cells), yeast, bacterial and mammalian artificial chromosomes (YAC, BAC and MAC, respectively), and other autonomously self-replicating sequences, although this is not required in all embodiments.

The targeting polynucleotides are contacted with the nucleic acid library under conditions that favor duplex formation as is outlined herein.

For cloning, preferred embodiments further comprise isolating the target nucleic acid. This is done as outlined herein, and frequently relies on the use of solid supports such as beads comprising a binding partner to the separation moiety; for example, antibodies (when antigens are used), streptavidin (when biotin is used), or as chemically derivatized particles, plates affinity matrix, non polar surface, ligand receptor, etc. In a preferred embodiment, the separation moiety is biotin and streptavidin coated microtiter plates or beads are used.

Alternatively, once a cloned gene is identified by the methods described herein or a gene sequence is otherwise identified, the gene sequence or a portion thereof may serve as the target DNA for generating a library of modifications, deletions or alterations to the targeted gene sequence by enhanced homologous recombination in a high throughput manner. Additionally, the library of modifications, deletions or alterations may be generated in an organism, such as Zebra Fish, having the targeted gene sequence in a high-throughput manner. One advantage to using Zebra Fish is that they are transparent and therefore amenable to a variety of optical screening procedures, as will be appreciated by the skilled artisan. By way of example the method employed by Module 2 may be modified as follows to generate a library of modified, or otherwise altered target genes in an organism:

1. The user generates, synthesizes or otherwise obtains a library of targeting polynucleotide sequences each having at least one homology clamp substantially complementary to a portion of the targeted gene sequence, as described above. Each member of the library contains varying alterations substitutions or deletions of nucleotides as compared to the targeted gene sequence, such that enhanced homologous recombination between a library member and the targeted gene sequence would result in the desired deletion, alteration or substitution within the targeted gene sequence. The library of targeting polynucleotide(s) is placed into solution, separately or mixed together, at an appropriate concentration, as is known in the art.

2. The user sets up the deck of the liquid handler by placing the different components (targeting polynucleotide(s), recombinase coating solution, deproteinization solution, PMSF, wash buffer etc.) in the liquid handler. For preparation of the coating solution, for each reaction 6 ml of the 5×coating buffer (50 mM Tris-acetate, pH 7.5, 250 mM sodium-acetate, 10 mM Mg-Acetate, and 5 mM DTT), 3.7 ml of 16.2 mM ATPgS (Boehringer Mannheim), and 0.7 ml of 1 mg/ml RecA (Promega) protein (total of 10.4 ml per reaction) are combined in a single tube which is placed in a 4° C. cooled position of a reagent rack on the robot deck. For preparation of the deproteinization solution, for each reaction, 0.6 ml of SDS solution (10 mg/ml) and 0.4 ml of Proteinase K (Boehringer Mannheim) are combined in a single 0.5 ml microfuge tube and placed in the reagent rack.

3. The liquid handler dispenses 5 ml of each targeting polynucleotide library solution into wells in a microtiter PCR plate. Alternatively each library member may be placed in a separate well, in which case the library members would not be placed in a common solution in step 2.

4. A robotic arm moves the plate to a plate sealer that seals the plate, and then into a thermal cycler block.

5. The thermal cycler heats the samples to 95° C. for 3 minutes, and then chills them to 4° C. for 5 minutes. As will be appreciated by those in the art, other types of denaturing may be done, for example chemical denaturants may be used. In addition, all subsequent steps may be done at room temperature.

6. A robotic arm removes the plate from the thermal cycler block, and returns it to the plate piercer, which pierces the seal.

7. The liquid handler transfers 3 ml from the content of the wells into a new plate. The liquid handler dispenses 10.4 ml of the coating mixture into each well, the mixture is mixed, preferably by pipetting up and down, and the plate is transferred to a thermally controlled position, where it is incubated at 37°0 C. for 15 minutes.

8. The liquid handler dispenses target DNA (5 mg in a volume of 5 ml) and 1.2 ml of 200 mM Mg-Acetate into the wells with the nucleoprotein filaments, the contents of the wells are mixed by pipetting up and down, and the mixture incubated further for 20 minutes.

9. The nucleoprotein filaments are microinjected, or otherwise introduced as known in the art, into oocytes of an organism (such as Zebra Fish for example) possessing the gene sequence of interest. Enhanced homologous recombination occurs within the oocytes, as described herein, and the resulting organisms are screened for phenotypic changes resulting from enhanced homologous recombination between the targeted gene sequence and a member from the targeting polynucleotide library.

Module 3: Transformation and Amplification of Clone DNA in Cells

Once the target DNA is captured, it can be amplified in bacteria. For this purpose, the captured DNA can be either transformed into chemically competent E. coli cells or electroporated into electro-competent E. coli cells. As will be appreciated within the art other cell types as outlined herein may be used. In the preferred embodiment, chemical transformation is totally automated using a liquid handler, an integrated thermal cycler, a barcode reader and applicator, a refrigerated plate position and a robotic plate handler.

In a preferred embodiment, a robotic station for Module 3 includes a micro-processor controlled liquid handler with a multi-channel pipettor head, equipped with a robotic arm for moving plates between deck positions (examples include, but are not limited to, Tecan Genesis, Beckman Coulter Biomek 2000, Beckman Coulter Biomek FX). The microprocessor runs a managing software program that coordinates the different components of Module 3, as described above for Modules 1 and 2. The liquid handler includes an integrated thermal cycler block on the deck, or a thermal cycler can be integrated with the liquid handler by the means of a robotic arm (examples include, but are not limited to, MJ Research DNA Engine and DNA Tetrad thermal cyclers, MWG Primus thermal cycler, and any other thermal cycler with a motorized lid that can be controlled remotely). The thermal cycler is controlled by the managing software. The system also includes a chilled plate position.

Referring to FIG. 11 the steps of a preferred embodiment of Module 2 follow:

1. The user sets up the deck of the liquid handler by placing the different reagents and samples on the deck in the appropriate positions.

2. The liquid handler dispenses 50 ml of competent suitable E. coli cells (for example, strain DH10B) into the wells of a 96-well PCR plate which is kept at a chilled position.

3. The liquid handler dispenses up to 3 ml of the captured DNA solution from Module 2 per well, and mixes it, preferably by pipetting up and down.

4. The plate is kept chilled for 30 minutes.

5. The robotic arm moves the plate into the thermal block which is at approximately 42° C., and keeps it there for 45 seconds. It then removes the plate and returns it to the chilled position for 3 minutes.

6. The liquid handler dispenses 950 ml of SOC medium into deep well 96-well plate.

7. The liquid handler aspirates the transformed cells and transfers them into the deep well plate.

8. The deep well plate is then removed from the robot deck and incubated at 37° C. for 1 hour, with optional shaking.

9. The cultures are plated on agar plates containing the appropriate agar and antibiotic selection, preferably OmniTray plates (Nunc) with LB medium and appropriate selection. Cultures are ideally evenly spread on the agar with the aid of glass beads.

10. The plates are incubated overnight at 37° C.

Alternatively, the captured DNA (2 ml) is electroporated into suitable E. coli competent cells (40 ml) using an electroporator (for example BTX Electro Cell Manipulator 600) in a manual process. The automation of electroporation is hampered at this stage by the low efficiency of currently available 96-well electrodes.

Those in the art will appreciate that there are a number of bacterial strains (and in some cases other procaryotic and eucaryotic cell may be used) that can be used for the purpose of transformation or electroporation of DNA and its propagation. Suitable strains include, but are not limited to, E. coli strains DH5a, DH10B, HB101, JM109, as well as other strains of bacteria, such as Bacillus subtilis. There are also many ways of preparing competent cells, such as calcium chloride, cobalt chloride, rubidium chloride, etc. (Maniatis et al., Molecular Cloning: A Laboratory Manual (1989), 2nd Ed., Cold Spring Harbor, N.Y. Guide to Molecular Cloning Techniques (1987), Academic Press, Inc., San Diego, Calif.). In addition, as will be appreciated by those skilled in the art, cells genetically engineered to contain reporter and selection genes, including green fluorescent protein (derivatives thereof) and drug selection genes may be used in accordance with the present invention.

Module 4: Clone Verification (Screen by Colony Picking and PCR)

In the preferred embodiment, the resultant colonies are screened by PCR to confirm the presence of the target clone DNA. The first step is the automated picking of colonies from the agar plates into microtiter plates filled with liquid medium, and incubation of these plates to allow growth of the cultures. Following this step, the cultures are used as templates in PCR reactions using the verification primers (described in Module 1). If there is a need to screen more than one plate (384 cultures) for one gene, pooling of plates is possible. If pooling is desired, multiple culture plates (inoculated with clones containing the same putative DNA target) are pooled into a single plate by the liquid handler by means of pipetting 10 ml of each well into a well of the same position in the pool plate, and the pooled cultures are used as templates for a first round of PCR analysis. The products of these PCR reactions are then analyzed by spectrofuorometric measurement using the dye PicoGreen. The wells that contain high concentration of DNA (which correspond to a successful PCR amplification, verifying the presence of the target sequence within the culture) are identified, and the cultures used as templates for the reactions in these wells are then re-inoculated into deep well 96-well plates containing 1 ml of appropriate medium. These plates are then grown to generate sufficient amount of cells for plasmid extraction. In case of pooling, after identification of positive pools, all the cultures that were pooled to create the positive pools are transferred by the liquid handler to a new plate, and new PCR reactions are set using these culture as templates. The results of this second round of PCR are analyzed by PicoGreen in the same manner as described above, and the positive individual cultures are inoculated into deep well plates as described.

In a preferred embodiment, a robotic station for Module 4 includes two micro-processor controlled liquid handlers with a multi-channel pipettor head, ideally one with an 8 channel pipettor head (examples include, but are not limited to, Tecan Genesis, Beckman Coulter Biomek 2000, Beckman Coulter Biomek FX with a SPAN8 arm), and one with 96 or 384 channels (examples include, but are not limited to, Tecan GenMate, Tomtec Quadra 96, and Beckman Coulter Biomek FX), both equipped with a robotic arm or other plate transport for moving plates between deck positions. The microprocessor runs a managing software program that coordinates the different components of Module 4, as described above for Modules 1-3. Again it will be appreciated that the managing software program and the microprocessor may different for each module, the hierarchy and location and number of microprocessors is not critical to the invention. The system includes multiple thermal cyclers blocks which are integrated with the system by the means of a robotic arm (examples include, but are not limited to, MJ Research DNA Tetrad thermal cyclers, MWG Primus thermal cycler, and any other high capacity thermal cycler with a motorized lid that can be controlled remotely). The thermal cyclers are controlled by the manage software program. The system also includes a plate sealer, a plate piercer, a plate reader capable of fluorescence measurements (examples include, but are not limited to, Tecan SpectraFluor and SpectraFluor Plus, BMG FluoStar Galaxy, Perkin Elmer LS and Molecular Devices SPECTRAmax Gemini XS), plate hotels, a plate filler, a barcode reader and applicator, and a colony picker. The components are integrated by means of a robotic arm or other plate transport mechanism.

Module 4 also includes a colony picker, preferably one that can pick colonies for extended periods of time unattended. Examples of colony pickers include, but are not limited to, GeneMachines Mantis, Autogen Autogenesys, Genetix Q-Bot and Q-Pix and Genomic Solutions Flexys.

Referring to FIGS. 12 and 13 the steps of a preferred embodiment of Module 4 follow:

1. 384-well barcoded culture plates are filled with 50 ml LB medium containing the appropriate antibiotic by a plate filler.

2. The agar plates containing the colonies (such as the OmniTrays), and the pre-filled culture plates are setup on the plate hotels of the colony picker (such as the Gene Machines Mantis), and the colonies are picked into the culture plates automatically by the colony picker. Several wells in each plate are left empty for different controls. The barcodes are entered into a database.

3. The inoculated plates are incubated at 37° C. overnight with optional shaking.

4. The user sets up the deck of the 96 or 384 channel liquid handler by placing the different reagents and samples on the deck in the appropriate positions.

5. If pooling is required, the liquid handler transfers 10 ml from each well in the pooled plates into the same position in the pool plate. The barcodes of all plates are entered into the database.

6. The liquid handler set up PCR reactions in 384-well PCR plates, using 1 ml from the culture plate(s) as templates and the verification primers (see module 1) for amplification primers.

7. The robotic arm transfers the plate to the plate sealer and the plate is sealed.

8. The robotic arm transfers the plate to the thermal cycler blocks.

9. The managing software closes the lids of the PCR blocks and starts the PCR program.

10. When the PCR amplification is complete, the lids are opened, and the robotic arm transfers the plate to the plate piercer, where the seal is pierced.

11. The robotic arm transfers the plate to the deck of the 96 or 384 channel liquid handler.

12. The liquid handler fills a black 384-well plate with 50-80 ml of PicoGreen reagent (10 mM Tris-HCl, pH 7.5, 1 mM EDTA, and 1/400 dilution of commercial PicoGreen from Molecular Probes), aspirates 1 ml of PCR product from each well, and dispenses it into the black plate. It fills certain wells with DNA of pre-determined concentration.

13. The robotic arm transfers the black plate into a plate reader, which excites the plate at 485 nm, and measure emission at 535 nm.

14. The measurement results are transferred to a software module that calculates a standard curve based on the control wells, converts the measurements to DNA concentrations, and determines which wells contain DNA with a concentration above a certain threshold. The threshold is determined based on the standard curve. The results are entered into the database.

15. If no pooling was used, the last step provided identification of individual positive cultures. If pooling was used, the 8-channel liquid handler transfers the original wells that were used to create the positive pools to a new plate. The changes in barcode and well position of these cultures are recorded in the database. The new plate is then used for a second round of PCR and PicoGreen analysis, which results in identification of individual positive cultures.

16. The 15 individual positive cultures, which generated the highest DNA concentrations in the PicoGreen assay, are inoculated into deep 96-well plates containing 1 mL of TB (terrific broth) medium containing the appropriate antibiotic and shaken overnight at 37° C.

As those in the art will appreciate, in a case when a gene is represented with very low abundance in the library, one round of capture may not provide sufficient enrichment for the gene to be cloned. In such a case, the cells can be harvested after the first round of target capture and transformation, and the plasmid DNA is purified in batch from the total of harvested cells using plasmid purification systems. This DNA is screened by PCR to verify the presence of the desired target sequence and then used in a second round of target capture.

Module 5: DNA Purification

In the preferred embodiment, the plasmid DNA is isolated from the cells from Module 4 with sufficient purity for subsequent restriction digest and sequence analysis. As those in the art can appreciate, there are many methods for isolating plasmid DNA from cells grown in 96-well microplates, including but not limited to magnetic beads (MagnaSil, Promega), and filter plates (Wizard SV96 kits, Promega; QIAprep 96 Turbo or R.E.A.L. Prep 96, Qiagen; PERFECTprep-96 VAC, Eppendorf-5 Prime, Inc.). Plasmid preparations can be performed on liquid handlers with plate handlers, magnetic positions, filter stations, tip washers, shakers, and plate hotels. As those in the art can appreciate, there are also commercial robots available that are sold for the sole purpose of performing plasmid preparations in high throughput (BioRobot 8000, Qiagen; Eppendorf 5-Prime Inc and Zymark; KingFisher, Labsystems).

In a preferred embodiment, the plasmids are purified using magnetic beads (for example, Promega's MagneSil technology). The system includes a microplate centrifuge and a robotic station. The robotic station for Module 5 includes a micro-processor controlled liquid handler with a multi-channel pipettor head, equipped with a robotic arm or other plate transport for moving plates between deck positions (examples include, but are not limited to, Tecan Genesis, Beckman Coulter Biomek 2000, Beckman Coulter Biomek FX). The microprocessor runs a managing software program that coordinates the different components of Module 5, as described above for Modules 1-4. The liquid handler includes a magnetic bead processing unit, which consists of a magnetic position, a barcode reader and applicator and a shaker. The components are integrated by means of a robotic arm or other plate transport mechanism. It is again emphasized that the microprocessor for the different modules may be different or the same and the managing software program may be the same or separate programs for each module.

Referring to FIG. 15 the steps of a preferred embodiment of Module 5 follow:

1. The user sets up the deck of the liquid handler with the reagents and samples in the appropriate deck positions.

2. The cultures in the deep 96-well plates are spun in a centrifuge for 10 minutes at 1200×g. The supernatant is decanted, and the plate is placed on the deck of the liquid handler.

3. The liquid handler dispenses 90 ml of Cell Resuspension Buffer to each well.

4. The robotic arm moves the plate to a shaker, and the pellet is completely resuspended.

5. The liquid handler dispenses 120 ml of Cell Lysis Buffer directly to the resuspended cells in each well. The plate is shaken for 3 minutes.

6. The liquid handler dispenses 120 ml of Cell Neutralization Buffer to each well. The plate is shaken for 5 minutes.

7. The liquid handler dispenses 25 ml of Clearing Resin to each well. The plate is shaken for 3 minutes.

8. The liquid handler transfers the lysate/ resin mix to a new 96-well plate on the magnetic position. The plate is left in this position for 1 minute for the resin to settle.

9. The liquid handler transfers 140 ml of the cleared lysate to a fresh plate (the binding plate) which is positioned on the shaker position.

10. The liquid handler dispenses 25 ml of binding resin into each well of the binding plate. The plate is shaken for 3 minutes.

11. The robotic arm transfers the plate to the magnetic position. The plate is left in this position for 1 minute for the resin to settle.

12. The liquid handler aspirates the supernatant.

13. The liquid handler transfers the remainder of the cleared lysate (140 ml) of the cleared lysate to the binding plate.

14. Repeat steps 10-12.

15. The liquid handler dispenses 100 ml of 80% Ethanol to each well in the Binding Plate.

16. The plate is shaken for 1 minute.

17. The robotic arm moves the plate to a shaker, and the plate is shaken for 1 minute.

18. The robotic arm transfers the plate to the magnetic position. The plate is left in this position for 1 minute for the resin to settle.

19. The liquid handler aspirates the supernatant.

20. Repeat steps 15-19 for a total of three washes.

21. Plate is left to dry for 10 minutes.

22. The liquid handler dispenses 100 ml of elution solution.

23. The robotic arm moves the plate to a shaker, and the plate is shaken for 3 minutes.

24. The robotic arm transfers the plate to the magnetic position. The plate is left in this position for 1 minute for the resin to settle.

25. The robotic arm transfers the eluant to a fresh plate.

Module 6: Restriction Analysis

In the preferred embodiment, the DNA is analyzed by restriction enzyme digestion to identify the sizes of the individual cDNA clones. The DNA digestion is performed by a liquid handler. Following the digestion, the DNA is loaded into an agarose gel for electrophoresis, and the gel is electrophoresed and inspected visually.

In a preferred embodiment, the robotic station for Module 6 includes a micro-processor controlled liquid handler with a multi-channel pipettor head, equipped with a robotic arm or other plate transport for moving plates between deck positions (examples include, but are not limited to, Tecan Genesis, Beckman Coulter Biomek 2000, Beckman Coulter Biomek FX). The microprocessor runs a managing software program that coordinates the different components of Module 6. The liquid handler includes an integrated thermal cycler block on the deck, or a thermal cycler can be integrated with the liquid handler by the means of a robotic arm (examples include, but are not limited to, MJ Research DNA Engine and DNA Tetrad thermal cyclers, MWG Primus thermal cycler, and any other thermal cycler with a motorized lid that can be controlled remotely). The thermal cycler is controlled by the managing software. The system also includes a chilled plate position, a barcode reader and applicator, and a gel loading device.

Referring to FIG. 15 the steps of a preferred embodiment of Module 6 follow:

1. The user sets up the deck of the liquid handler with the reagents and samples in the appropriate deck positions. Plasmid DNA is present in a 96-well plate format. Appropriate restriction enzyme mixes consisting of water, buffer and enzymes are prepared by the user in microfuges, and kept in a refrigerated position on the deck. The configuration of the restriction setup is entered into the database.

2. The liquid handler aspirates the mix and dispenses it into each well of a fresh 96-well PCR plate.

3. The liquid handler adds DNA to each well and mixes it by pipetting up and down.

4. The managing software starts a program on the thermal cycler that keeps it at 37° C.

5. The robotic arm moves the plate into the thermal cycler block.

6. The managing software closes the lid. After 90 minutes the lid is opened, and the robotic arm removes the plate and places it on the deck.

7. The user places an agarose gel containing ethidium bromide on the gel loading fixture.

8. The liquid handler aspirates loading dye and dispenses it into the restriction digests.

9. The liquid handler aspirates the digested DNA:loading dye mixture and loads it into the wells of the gel.

10. The user transfers the gel to an electrophoresis chamber and electrophoreses the DNA under the appropriate voltage for the appropriate amount of time to obtain ideal resolution of the DNA fragments.

11. The gel is placed on a UV illuminator, and the digital image of the gel is obtained and stored in the database.

A simplified flow chart of these processes is provided in FIG. 15.

Module 7: Sequencing

In the preferred embodiment, the DNA purified in Module 5 is also used for sequence analysis. A liquid handler sets up sequencing reactions using a primer used for making the targeting polynucleotide or for verification (see Module 1). If the clone is guaranteed to be full-length, it is also sequenced with 5′ and 3′ vector primers. Each clone is sequenced with 1 or 3 primers. As those in the art can appreciate, multiple chemistries are available for sequencing plasmid DNA, for example the BigDye chemistry of PE Biosystems, and the WellRED chemistry of Beckman-Coulter. There are multiple chemistries for purifying sequencing reactions for analysis, including filter plates, magnetic bead purification, columns etc. Accordingly, there are multiple detection systems for analyzing the sequencing reactions, including different instruments by ABI, Beckman Coulter and Molecular Devices. In this preferred embodiment, the process described includes BigDye chemistry, MagneSil based purification, and capillary electrophoresis by the ABI PRISM 3100 DNA sequencer. However, the process can be performed by any other combination of chemistry, purification and sequencing aparatus.

In a preferred embodiment, the robotic station for module 6 includes a micro-processor controlled liquid handler with a multi-channel pipettor head, equipped with a robotic arm or other plate transport for moving plates between deck positions (examples include, but are not limited to, Tecan Genesis, Beckman Coulter Biomek 2000, Beckman Coulter Biomek FX). The microprocessor runs managing software program that coordinates the different components. The liquid handler includes an integrated thermal cycler block on the deck, or a thermal cycler can be integrated with the liquid handler by the means of a robotic arm (examples include, but are not limited to, MJ Research DNA Engine and DNA Tetrad thermal cyclers, MWG Primus thermal cycler, and any other thermal cycler with a motorized lid that can be controlled remotely). The thermal cycler is controlled by the managing software. The system also includes a plate sealer, a plate piercer, a barcode reader and applicator, and a magnetic bead processing unit, or a vacuum filtration unit. The components are integrated by means of a robotic arm or other plate transport mechanism.

Referring to FIG. 15 the steps of a preferred embodiment of Module 7 follow:

1. The user sets up the deck of the liquid handler with the reagents and samples in the appropriate deck positions. Plasmid DNA is present in a 96-well plate format.

2. The liquid handler set up the sequencing reaction by mixing template DNA, primer, and sequencing mix in a fresh plate.

3. The robotic arm transfers the plate to the plate sealer, where it is sealed.

4. The robotic arm transfers the plate into the thermal cycler block.

5. The managing software closes the lid and starts the cycling program.

6. At the end of the cycling program the lid is opened and the plate is transferred to the plate piercer, where the seal is pierced.

7. The liquid handler adds 180 ml pf Magnesil™ BigDye Terminator Sequencing Reaction Cleanup Resin to each well.

8. The mixture is mixed by pipetting up and down.

9. The robotic arm transfers the plate to the magnetic position.

10. The liquid handler aspirates and discards the supernatant.

11. The robotic arm transfers the plate to a non-magnetic position.

12. The liquid handler dispenses 100 ml of wash solution and mixes by pipetting up and down.

13. The robotic arm transfers the plate to the magnetic position.

14. The liquid handler aspirates and discards the supernatant.

15. Steps 12-14 are repeated for a total of two washes.

16. The plate is left to dry for 5 minutes.

17. The robotic arm transfers the plate to a non-magnetic position.

18. The liquid handler dispenses 6-20 μl formamide. Plate is incubated for 2 minutes.

19. The robotic arm transfers the plate to the magnetic position.

20. The liquid handler transfers the supernatant to a clean plate.

21. The user removes the plate and loads it on an ABI PRISM 3100 DNA sequencer.

As will be appreciated by those in the art, when a target gene is isolated, it may be that the isolated target sequence is not a full-length gene: that is, it does not contain a full open reading frame. In this case, either the experiments can be run again, using either the same targeting polynucleotides or targeting polynucleotides based on some of the newly obtained sequence with the same or new libraries. Another possibility is to screen more of the colonies that have been isolated. In addition, multiple experiments may be run to enrich for the desired target sequence. For instance, multiple 5′ and 3′ derived probes can be used in succession to obtain full-length gene clones.

Module 8: Gene Database

As will be appreciated by those in the art, the use of a computer database greatly facilitates the storage, manipulation and retrieval of the large amount of information generated during the automated cloning procedure. During all steps of this process, as has been illustrated in the description of the different modules, data concerning sequences, primers, microplate barcodes, position of particular samples within a microplate, and information about particular assays is entered into the database. General information about the location of reagents including, but not limited to, DNA libraries, oligonucleotides, enzymes etc. is also stored in the database. Digital images of electrophoresis gels and other visual information are stored in the database as well.

As will be appreciated by those in the art, there are many different types of databases that can be used. In the preferred embodiment, the database is Oracle. Additional custom software is written to facilitate data entry into the database using a user-friendly network based interface. The microprocessors running the automation equipment have access to the database, and retrieve the information that is required for the different assays from the database, by the means of a script that retrieves information from the database and converts it into an ASCII file. The microprocessors also output data in the form of ASCII files, which is converted to database format and imported into the database.

An Additional Module for Screening Cells or Organisms for Genetic Modifications

Screening for cells can be automated as those in the art can appreciate. There are many liquid handlers, robotic arm systems, etc that are used for cell culturing.

In some preferred embodiments, the instrumentation includes a microscope(s) with multiple channels of fluorescence; plate readers to provide fluorescent, ultraviolet and visible spectrophotometric detection with single and dual wavelength endpoint and kinetics capability, fluroescence resonance energy transfer (FRET), luminescence, quenching, two-photon excitation, and intensity redistribution; CCD cameras to capture and transform data and images into quantifiable formats; and a computer workstation. These will enable the monitoring of the size, growth and phenotypic expression of specific markers on cells, tissues, and organisms; target validation; lead optimization; data analysis, mining, organization, and integration of the high-throughput screens with the public and proprietary databases.

These instruments can fit in a sterile laminar flow or fume hood, or are enclosed, self-contained systems for cell culture growth and transformation in multi-well plates or tubes and for hazardous operations. The living cells will be grown under controlled growth conditions, with controls for temperature, humidity, and gas for time series of the live cell assays.

Flow cytometry or capillary electrophoresis formats can be used for individual capture of magnetic and other beads, particles, cells, and organisms.

Phenotypic Modification and Analysis

Once variant target sequences are made, any number of different phenotypic screens may be done. As will be appreciated by those in the art, the type of phenotypic screening will depend on the mutant target nucleic acid and the desired phenotype; a wide variety of phenotypic screens are known in the art, and include, but are not limited to, phenotypic assays that measure alterations in multicolor fluorescence assays; cell growth and division (mitosis: cytokinesis, chromosome segregation, etc); cell proliferation; DNA damage and repair; protein-protein interactions, include interactions with DNA binding proteins; transcription; translation; cell motility; cell migration; cytoskeletal (microtubule, actin, etc) disruption/localization; intracellular organelle, macromolecule, or protein assays; receptor internalization; receptor-ligand interactions; cell signalling; neuron viability; endocytic trafficking; cell/nuclear morphology; activation of lipogenesis; gene expression; cell-based and animal-based efficacy and toxicity assays; apoptosis; cell differentiation; radiation resistance/sensitivity; chemical resistance/sensitivity; permeability of drugs; pharmocokinetics; pharmacodynamics; pharmacogenomics in cells and animals; nucleus-to-cytoplasm translocation; inflammation-inflammatory tissue injury; wound healing; cell ruffling; cell adhesion; drug induced redistribution of target protein; immunoassays for diagnostics and the emerging field of proteomics.; cell sorting; phenotypic screening of cells and animals; phenotyping small molecule drug inhibitors; biovalidation of drug targets in transgenic recombinant cell and animal phenotypes; single and multiple nucleotide polymorphisms diagnostics; loss of heterozygosity (loh) and other chromosomal aberration diagnostics; in situ gene targeting (hybridization) in cells, tissues, and animals; in situ gene recombination in cells and animals; and gene delivery and therapy. See Keller, Current Opin. In Cell Biol. 7:862 (1995); Hsin et al., Nature 399(6743):362 (1999); Giuliano et al., Tibtech 16:135 (1998); Conway et al., J. Biomolecular Screening 4:75 (1999); Giulano et al., J. Biomolecular Screening 2:249 (1997); Forrester et al., Genetics 148:151 (1998); Reiter et al., Genes Dev. 13:2983 (1999); Carmeliet et al., Nature 380:435 (1996); Ferrara et al, Nature 380:439 (1996); Hidaka et al., Genetics 96:7370 (1999); DeWeese et al., Medical Sci. 95:11915 (1998); Aszterbaum et al., Nature Med. 5:1285 (1999); Abuin et al., Mol. Cell. Biol. 20:149 (2000); de Wind et al., Nature Genetics 23:359 (1999); Gailani et al., Nature Genet. 14:78 (1996); Tanzi et al., Neurobiol. Dis. 3:159 (1996); Jensen et al., Artherosclerosis 120:57 (1996); Lipkin et al., Nature Genetics 24:27 (2000); Chen et al., Genes Dev. 11:2958 (1997) and Brown et al., Genes Dev. 11:2972 (1997); and and U.S. Pat. Nos. 5,989,835 and 6,027,877.

In a preferred embodiment, the compositions and methods of the invention can be used in screening variant target sequences in the presence of candidate agents. By “candidate bioactive agent” or “candidate drugs” or grammatical equivalents herein is meant any molecule, e.g. proteins (which herein includes proteins, polypeptides, and peptides), small organic or inorganic molecules, polysaccharides, polynucleotides, etc. which are to be tested against a particular target. Candidate agents encompass numerous chemical classes. In a preferred embodiment, the candidate agents are organic molecules, particularly small organic molecules, comprising functional groups necessary for structural interaction with proteins, particularly hydrogen bonding, and typically include at least an amine, carbonyl, hydroxyl or carboxyl group, preferably at least two of the functional chemical groups. The candidate agents can interact with nucleic acids to prevent gene expression. The candidate agents often comprise cyclical carbon or heterocyclic structures and/or aromatic or polyaromatic structures substituted with one or more chemical functional groups.

Candidate agents are obtained from a wide variety of sources, as will be appreciated by those in the art, including libraries of synthetic or natural compounds. As will be appreciated by those in the art, the present invention provides a rapid and easy method for screening any library of candidate agents, including the wide variety of known combinatorial chemistry-type libraries.

In a preferred embodiment, candidate agents are synthetic compounds. Any number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods. In a preferred embodiment, the candidate bioactive agents are organic moieties. In this embodiment, as is generally described in WO 94/24314, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

Alternatively, a preferred embodiment utilizes libraries of natural compounds in the form of bacterial, fungal, plant and animal extracts that are available or readily produced, and can be tested in the present invention.

Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In a preferred embodiment, candidate bioactive agents include proteins, nucleic acids, and chemical moieties.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occuring proteins or fragments of naturally occuring proteins. Thus, for example, cellular extracts containing proteins, or random or directed digests of proteinaceous cellular extracts, may be attached to beads as is more fully described below. In this way libraries of procaryotic and eucaryotic proteins may be made for screening against any number of targets. Particularly preferred in this embodiment are libraries of bacterial, fungal, viral, and mammalian proteins, with the latter being preferred, and human proteins being especially preferred.

As will be appreciated by those in the art, it is possible to screen more than one type of candidate agent at a time. Thus, the library of candidate agents used in any particular assay may include only one type of agent (i.e. peptides), or multiple types (peptides and organic agents).

The candidate agents are added to the screens under reaction conditions that favor agent-target interactions. Generally, this will be physiological conditions. Incubations may be performed at any temperature which facilitates optimal activity, typically between 4 and 40 C. Incubation periods are selected for optimum activity, but may also be optimized to facilitate rapid high through put screening. Excess reagent is generally removed or washed away.

A variety of other reagents may be included in the assays, or other methods of the invention. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### Example 1

### High Throughput Semi-Automated Gene Cloning

Semi-automation includes automated, parallel processing of the targeting and capture reactions between affinity labeled cssDNA probes and homologous DNA targets, which are a subset of the robotic functions listed in the “Full Automation of Gene Targeting Applications” in Example 1 described above. Semi-automation has increased the throughput of cloning by 100-1000 fold over manual methods.

Comparison Between the Manual and Automated Targeting and Capture Reactions Isolation of Clones from Simple DNA Libraries

Sample RecA-mediated cloning results are easily quantified by examining data from a control library. These libraries are made by mixing a defined ratio of two plasmids, pHPRT and pUC. The rare plasmid (pHPRT) contains a 530 bp region of the HPRT gene inserted into the b-galactosidase gene and the abundant plasmid pUC carries a native b-galactosidase gene (pUC). The probe in all reactions is homologous to the HPRT region in the rare plasmid. The ratio of pHPRT:pUC in the library was 1:10,000, which represents the frequency of an abundant gene in a cDNA library.

TABLE 1

| | Manual | |
|---|---|---|
| | Capture (%) | Automated Capture (%) |
| First Round Capture of | 2 | 1.35 |
| pHPRT clones | | |
| Second Round Capture of | 76 | 59 |
| pHPRT clones | | |

A 318 bp biotin-HPRT probe was coated with recombinase and targeted to the control library. Positive colonies were rapidly screened by visualization of white colonies carrying the pHPRT plasmid or blue colonies carrying the pUC plasmid when plated on the chromogenic substrate 5-bromo-4-chloro-indolyl-D-b-galactoside (X-gal).

Primers used to generate 318 bp biotinylated HPRT probe for clone isolations:

| | |
|---|---|
| hExo3-2A | 5′ ATCACAGTTCACTCCAGCCTC 3′ |
| h/m300B | 5′ TATAGCCCCCCTTGAGCACACAG 3′ |

The efficiency of isolation of the pHPRT plasmid from a control library was similar for the manual and automated captures. After two rounds of capture, the majority of the resulting colonies contained the desired pHPRT plasmids after targeting, capture, washing, elution, and transformation of the selected sample. Thus, only relatively few colonies need to be analyzed to identify the desired clone.

### Example 2

### Gene Family and Inter-Species Cloning

### A. Mouse Actin Gene Family cDNA Cloning Using a Human Beta Actin Probe

The recombinase-mediated targeting and clone isolation technology was used to isolate multiple sequence variants of the mouse actin gene family using a DNA probe containing the human b-actin sequence.

Sequence of 512 base pair human beta actin probe used in RecA protein-mediated mouse cDNA isolation:

GACTACCTCATGAAGATCCTCACCGAGCGCGGCTACAGCTTCACCACCAC
GGCCGAGCGGGAAATCGTGCGTGACATTAAGGAGAAGCTGTGCTACGTCG
CCCTGGACTTCGAGCAAGAGATGGCCACGGCTGCTTCCAGCTCCTCCCTG
GAGAAGAGCTACGAGCTGCCTGACGGCCAGGTCATCACCATTGGCAATGA
GCGGTTCCGCTGCCCTGAGGCACTCTTCCAGCCTTCCTTCCTGGGCATGG
AGTCCTGTGGCATCCACGAAACTACCTTCAACTCCATCAGAAGTGTGACG
TGGACATCCGCAAAGACCTGTACGCCAACACAGTGCTGTCTGGCGGCACC
ACCATGTACCCTGGCATTGCCGACAGGATGCAGAAGGAGATCACTGCCCT
GGCACCCAGCACAATGAAGATCAAGATCATTGCTCCTCCTGAGCGCAAGT
ACTCGTGTGGATCGGCGGCTCCATCCTGGCCTCGCTGTCCACCTTCCAGC
AGATGTGGAT

TABLE 3
Heterologies between Human Beta Actin and Mouse Actin Family
members

| | Percent heterology between mouse |
|---|---|
| | actin and Human Beta Actin (%) |
| Mouse beta actin | 9 |
| Mouse cytoskeletal gamma actin | 11 |
| Mouse skeletal muscle actin | 15 |
| Mouse vascular smooth muscle actin | 17 |

Primers used to synthesize the biotinylated human actin probe

| | |
|---|---|
| Actin1: | 5′ ACGGACTACCTCATGAAGATCC 3′ |
| Actin2: | 5′ ATCCACATCTGCTGGAAGGTG 3′ |

In the gene cloning procedure, biotin-labeled cssDNAs were denatured and coated with RecA recombinase protein. These nucleoprotein filaments were targeted to homologous target DNAs in a DNA library. The hybrids were deproteinized and captured on streptavidin-coated magetic beads. The homologous dsDNA target was eluted and transformed into bacteria. After recombinase-mediated targeting, clone capture, and DNA transformation into bacterial cells, the resulting colonies were screened by PCR, colony hybridization to filters, and DNA sequencing to identify the actin-related clones. Colony hybridization involved the transfer of the colonies from the plates to Hybond filters (Amersham), denaturation of the DNA, neutralization of the filters, and hybridization of a radiolabeled or biotinylated ssDNA probe to the positive clones. The desired clones were picked and cultured for DNA purification and sequencing. The use of recombinase-mediated homologous targeting has significant advantages over thermodynamically driven DNA hybridization such as PCR-based DNA amplification, which is widely used to isolate gene homologs and can have non-specific background hybridizations and artifacts due to improper renaturation of repeated sequences.

This example demonstrates that the recombinase-catalyzed cloning technology is not only a powerful method for isolation of related members of gene families but also allows cross-species gene cloning. Four mouse actin gene family members were isolated from the mouse embryo cDNA library using a human B-actin probe in RecA protein-mediated targeting reactions. The nucleotide sequence variation between the human B-actin probe and the mouse actin cDNAs ranged from 9-17%. The heterologies between the full length B-actin human actin cDNA and the mouse actin cDNAs were between 9-17%.

### B. Cross Species Cloning of Mouse Rad51A Using a Human Rad51A Probe

The human Rad51A probe was used to target and capture the mouse Rad51A cDNA from a complex mouse embryo cDNA library. The nucleotide sequence variation (heterology) between human Rad51A and mouse Rad51A is 10%.

Sequence ID#3. Sequence of human Rad51A biotinylated probe used to capture mouse Rad51A cDNA from mouse embryo cDNA library

ATTGACACTGAGGGTACCTTTAGGCCAGAACGGCTGCTGGCAGTGGCTGA
GAGGTATGGTCTCTCTGGCAGTGATGTCCTGGATAATGTAGCATATGCTC
GAGCGTTCAACACAGACCACCAGACCCAGCTCCTTTATCAAGCATCAGCC
ATGATGGTAGAATCTAGGTATGCACTGCTTATTGTAGACAGTGCCACCGC
CCTTTACAGAACAGACTACTCGGGTCGAGGTGAGCTTTCAGCCAGGCAGA
TGCACTTGGCCAGGTTTCTGCGGATGCTTCTGCGACTCGCTGATGAGTTT
GGTGTAGCAGTGGTAATCACTAATCAGGTG

Primers used to synthesize 329 bp biotinylated human Rad51A probe

| | |
|---|---|
| Rad51A-F689 | 5′ ATT GAC ACT GAG GGT ACC TTT AGG 3′ |
| Rad51A-R1017 | 5′ CAC CTG ATT AGT GAT TAC C 3′ |

After recombinase-mediated targeting, clone capture, and DNA transformation into bacterial cells, the resulting colonies were screened by PCR, colony hybridization to filters, and DNA sequencing to identify the Rad51A clones. Colony hybridization involved the transferof the colonies from the plates to Hybond filters, denaturation of the DNA, neutralization of the filters, and hybridization of a radiolabeled or biotinylated ssDNA probe to the positive clones. The desired clones were picked and cultured for DNA purification and sequencing. The recombinase-mediated targeting and capture is a powerful method toisolate interspecies DNA clones. The mouse Rad51A cDNA was cloned using a probe containing the human Rad51A sequence in RecA protein-mediated targeting and capture reactions.

### Example 3

### Gene Cloning by Amplification of DNA on Solid Matrices, e.g. Beads, Chips, Plates

Rare or limited nucleic acids have been amplified by transformation of the captured DNA into bacterial cells. As an alternative to amplifying in biological hosts, nucleic acids can be immobilized onto beads, chips, plates, optical fibers, or other solid supports and can be cloned by PCR or other duplication methods to potentially generate 104-108 copies of each cDNA clone or genomic fragment. Multiple sequence variants (gene families, polymorphic genomic fragments, etc. ) can be amplified in parallel on solid matrices and can be separated by fluorescent sorting methods, microarray matrices, etc and can be sequenced. Differentially expressed genes can be compared within one library or the expression of particular genes can be compared between libraries. Gene cloning and amplification will allow the identification of rarely expressed genes and the elucidation of single-nucleotide polymorphisms (SNP)-bearing fragments that are differentially represented from two populations of individuals. Additional applications include gene amplification (cloning); mutagenesis, modifications (mutations, gene duplications, gene conversion, etc), and evolution of genes; Isolation of gene families, gene orthologs, and paralogs; Differential gene expression; single and multiple nucleotide polymorphisms (genetic variation); genotyping and haplotyping; multigenic trait analysis and inference, allelic frequency; Association of alleles; Association of haplotypes with phenotypes (find trait-associated genes and trait associated polymorphisms); Identification of disease-associated alleles and polymorphisms; Linkage mapping and disequilibrium, Loss of heterozygosity (LOH) and other chromosomal aberration diagnostics; Single nucleotide polymorphism (SNP) validation; nucleic acid library production, subtraction and normalization; gene mapping; gene segregation analysis.

Gene Isolation and Nucleic Acid Cloning on the Solid Matrix

DNAs that have been isolated on solid supports such as beads, chips, filters and other supports in recombinase-mediated targeting reactions can be cloned (amplified) on/from the support. Nucleic acid probes that are immobilized on a solid matrix (beads, chips, filters, etc.). can be used to hybridize to specific target cDNA clones or genomic DNA fragments from simple or complex mixtures (libraries) of nucleic acids. To clone the desired target molecule, the cDNA or genomic DNA fragment is amplified directly on the solid support or is cleaved from the support and then amplified by PCR or other amplification methods. Recombinase-mediated hybridization increases the specificity and sensitivity of capture and amplification on beads.

Gene Cloning and Expression Profiling

The genomic DNA fragment encoding a desired differentially expressed gene can be isolated and cloned. Nucleic acids probes (oligonucleotides, PCR fragments) are first attached to solid matrices (beads, chips, filters, etc), coated with recombinase protein, and are used to capture target cDNAs from libraries. The expression levels of the cDNAs will be determined in two or more populations (of cells, tissues, etc). For example, to capture genomic DNA of a differentially expressed gene, the desired cDNA of an overexpressed or underexpressed gene that was captured on the solid matrix is coated with recombinase and is used as the probe to capture the genomic DNA fragment from a library (genomic, cell or tissue extract, etc). The desired genomic DNA is amplified on the solid matrix or is first cleaved from the matrix and then amplified.

Gene Cloning and Identification of DNA Sequence Polymorphisms

Related genes can be isolated using recombinase-mediated gene targeting and capture on solid supports. Libraries of nucleic acid molecules that contain polymorphic fragments specific to each population that is analyzed can be obtained. The sequence of each nucleic acid on the solid support can be determined and single and multiple polymorphisms can be identified.

Gene Cloning and Drug Screening

The desired cDNA or genomic fragment or other nucleic acid can be isolated on solid supports as described above using recombinase-mediated gene targeting. The In vitro transcription of the cDNA or gene can be performed on the solid matrix. In addition, in vitro translation of the resulting mRNA to protein can be performed on the solid matrix. The protein products derived from in in vitro transcription and translation can be used directly in compound and drug screening assays.

Gene Cloning, Protein Binding, and DNA Modification

Proteins that bind to the cloned DNA sequences can be identified and isolated. The desired cDNA or genomic fragment or other nucleic acid will be isolated on solid supports as described above using recombinase-mediated gene targeting. Cell extracts can be added to the solid supports that contain the cloned DNAs and the proteins that bind to the DNA can be identified and isolated. Alternatively, to modify (alkylate, nick, break, digest, etc) the cloned DNA, specific proteins can be used to modify the desired sequence.

### Example 4

### Biovalidation of Gene Targets by Phenotypic Screening

To generate mutant substrates for high throughput phenotyping, exact or degenerate EHR probes are used to generate a library of transgenic cells or organisms with single or multigene knockouts, corrections, or insertion of single nucleotide polymorphisms (SNPs) in organisms (such as zebra fish and C. elegans), totipotent cells (such as embryonic stem [ES] cells), proliferative primary cells (such as keratinocytes or fibroblasts), and transformed cell lines (such as CHO, COS, MDCK, and 293 cells). ES cells can be further differentiated into embryoid bodies, primitive tissue aggregates of differentiated cell types of all germinal origins, and keratinocytes can be induced to stratify and differentiate into epidermal tissue. DNA is delivered to cells using standard methods including lipofection, electroporation, microinjection, etc. mutagenized cells, tissues and organisms can be used for phenotypic and drug screening for validation of gene targets (see below). The high-throughput platform is designed to biovalidate gene targets by screening chemical or biological libraries that enhance or cause reversion of the phenotype. The high-throughput EHR phenotypic screening technology allows genetic profiling of compound libraries, selection of new drug leads, and identification and prioritization of new drug targets.

### A. Biovalidation of Aging Targets in Organisms and Cells

There are germline signals that act by modulating the activity of insulin/IGF-1 (insulin-like growth factor) pathway that are known to regulate the aging of C. elegans. It has been established that the insulin/IGF-1-receptor homologue, DAF-2, plays a role in signaling the animal's rate of aging since mutants with reduced activity of the protein have been shown to live twice as long as normal C. elegans. EHR introduces additional mutations into DAF-2, and identifies and/or isolate additional DAF-2 family members using a degenerate HMT, consisting of a recombinase-coated complementary single-stranded DNA consensus sequence. These experiments only extended to clone interspecific DAF-2 homologues, including zebrafish, mouse, and human. EHR used to disrupt DAF in zebrafish, and its effect on the aging process is assessed in the whole organism by screening for organisms with an extended lifespan. The same procedure modifies mouse or human DAF in primary cells, including keratinocytes or fibroblasts, and the proliferative capacity of cells is ascertained. Specific related genes are disrupted using EHR, or degenerate HMT probes are directly introduced into cells and animals to modify DAF-2-related genes, and aberrant phenotypes are analyzed.

EHR is also be used to generate Green Florescent protein (GFP) DAF-2 wild-type (WT) and mutant chimeras, and the subcellular localization of the proteins are determined. The genes of interest are biovalidated by screening for drugs that enhance or cause revert of the altered phenotype.

### B. Biovalidation of Neuronal Targets in Organisms

To understand the mechanisms that guide migrating cells, the embryonic migrations of the C. elegans canal-associated neurons (CANs) are analyzed. The ceh-10 gene specifies the fate of canal-associated neurons (CAN) in C. elegans. Mutations that reduce ceh-10 function result in animals with withered tails (Wit) which have CANs that are partially defective in their migrations. Mutations that eliminate ceh-10 function result in animals that die as clear larvae (Clr) who have CANs that fail to migrate or express CEH-23, a CAN differentiation marker. EHR technology is used to clone related genes using degenerate probes, and ablate or modify their function in C. elegans. EHR is used to isolate zebra fish ceh-10, and moderate to severe mutations of the protein is introduced into the organism to determine recombinants having a similar phenotype to Wit or Clr.

### C. Biovalidation of Cardiovascular Development Targets in Organisms, Tissues, and Cells

Gata5 is an essential regulator in controlling the growth, morphogenesis, and differentiation of the heart and endoderm in zebra fish. Gata5 is a master switch that induces embryonic stem cells to become heart cells. From loss-and gain-of function experiments, the zinc finger transcription factor Gata5 has been shown to be required for the production of normal numbers of developing myocardial precursors and the expression of normal levels of several myocardial genes in zebra fish. EHR is to clone related Gata5 family members (zebra fish, mouse and human), and is used to introduce additional mutations in Gata5 and its homologues in zebra fish. EHR is used to ablate or modify Gata5 function in mouse embryonic stem (ES) cells, which differentiate into embryoid bodies (EBs). ES cells are plated into duplicate wells to undergo differentiation into EBs, and one set are prescreened using immunoflorescence with antibodies to terminally differentiated gene products to eliminate EBs which undergo normal differentiation. EBs defective in terminal differentiation are disaggregated, replated, and cell sorted to score for cardiac cell populations to determine the effect of the targeted mutation on the differentiation process. Gene expression profiles are determined using microarrays, DNA chips, or related technologies. Cultured mutant EBs are used for drug screening. Additionally, with human embryonic stem cells, the same set of experiments can be repeated to determine if Gata5 plays a similar role in human tissue, and these and the mouse cultured mutant EBs can be used for drug screening.

### D. Biovalidation of Vascular and Hematopoietic Targets in Cells and Tissues

Heterozygous mutations Disruption of gene function from a single allele is adequate to cause a phenotype in cells for a subset of genes with tightly regulated abundance. In examples D-F, disruption of a single allele results in a screenable phenotype. Disruption of a single allele of either VEGF or GATA-1 in embryonic stem cells (ES cells) results in an easily identifiable phenotype upon differentiation of targeted cells into embryoid bodies (EBs) of lymphoid and endothelial origins (Keller and Orkin reviews). Degenerate homologous probes are utilized to identify other novel, related genes which function in a common pathway, and EHR is used to ablate or modify gene function. ES cells is differentiated into cells of lymphoid and endothelial origin, and screened in a similar manner to that of Gata5 mutants.

### E. Biovalidation of DNA Repair Targets

Disruption of a single allele of the mismatch repair gene, Msh2, in ES cells results in defective response to oxidative stress induced by low-level radiation [PNAS 1998 95(20)11915-20]. These cells have an increased survival in response to radiation through a failure to undergo apoptosis. Related genes are obtained using EHR with degenerate probes, and gene function is ablated or modified to screen for novel family members that also have the same defective response to oxidatitve stress. This is assessed by screening for survival of cells with damaged DNA resulting from apoptotic changes. In addition, EHR is used to disrupt Msh2 in both undifferentiated or stratified keratinocytes in order to mismatch repair operating through a common pathway in both cell types.

F: Disruption of a single allele of the human tumor suppressor gene, Patched (Ptch), [Nature Medicine November 1999 Volume 5, #11 pp. 1285-1291] results in a predisposition to basal cell carcinoma, the most prevalent form of cancer in humans, in mouse skin exposed to ultraviolet (UV) and ionizing radiation. EHR is used to disrupt Ptch and other genes in the hedgehog signaling pathway in cells (including human or mouse keratinocytes and fibroblasts). Both undifferentiated and differentiated cells are screened for changes induced by UV and ionizing radiation to determine that the phenotype of the whole organism is recapitulated.

### G. Biovalidation of DNA Repair Targets in Cells—Homozygous and Multiple Mutations

Some genes require disruption of multiple alleles in order to obtain a screenable phenotype, and in these instances we utilize cells with single or multiply disrupted alleles to perform mutagenesis using exact and/or degenerate EHR probes to determine other key players on a common pathway. We can use EHR is used to disrupt a single key component in the DNA damage response pathway, Rad 51A, and uses degenerate EHR probes to common functional domains, such as the ATP binding domain, to functionally modify radiation repair in cells such as ES cells, keratinocytes, and fibroblasts.

### H. Biovalidation of DNA Repair Targets in Cells—Trans-Dominant Mutations

Trans-dominant mutations have been shown to play a role in a large number of highly prevalent human diseases, including nevoid basal cell carcinoma syndrome (human Ptch), Alzheimer's disease (presenilin), cardiac hypertrophy (sarcomeric proteins), familial hypercholesterolemia (LDL receptor), obesity (melanocortin-4), and hereditary non-polyposis colon cancer (DNA mismatch repair genes MLH-1 and MLH-3). [Nature Genetics vol. 24 January 2000 pp 27-35] We use EHR to perform insertional mutagenesis to create germline trans-dominant mutations in cell lines (such as ES, fibroblasts, keratinocytes, or transformed cell lines) for a phenotype screen. EHR mutagenesis utilized to create dominant negative mutant forms of the DNA mismatch repair genes, MLH-1 and MLH-2, by creating truncations or chimeric truncation/GFP fusion proteins. These trans-dominant mutations can be expressed in cell lines (such as ES, fibroblasts, keratinocytes, or transformed cell lines), and the fluorescence tagged mutant protein is followed to determine which mutations disrupt specific cellular functions, including subcellular distribution or trafficking.

### I. Biovalidation of Signaling Pathways in Cells

EHR is utilized to insert GFP and/or other fluorescent tags into a single allele of the gene, or multiple genes, in a non-disruptive manner. Target genes are involved in important signaling pathways, such as the WNT/wingless, Hedgehog, or DNA repair pathways. EHR derived mutants or SNP containing proteins are generated to determine their effects on cellular function, including effects on subcellular localization, cell motility and migration, and cytoskeletal functions, etc.

### J. Biovalidation of Cell Growth Targets in Single-celled Organisms

Yeast Gic1 and Gic2 proteins are required for cell size and shape control, bud site selection, bud emergence, actin cytoskeletal organization, mitotic spindle orientation/positioning, and mating projection formation in response to mating pheromone. Each protein contains a consensus CRIB (Cdc42/Rac-interactive binding) motif and binds specifically to the GTP-bound form of Rho-type Cdc42 GTPase, a key regulator of polarized growth in yeast. Mutations are introduced into Gic1 or Gic2 in S. cerevisiae by EHR, and cells with aberrant growth phenotypes are identified. The genes are biovalidated by screening for drugs that enhance or cause reversion of the altered phenotype.

### K. Biovalidation of Hormone Receptors

Hormone receptors are excellent drug targets because their activity is important in intracellular signaling pathways. Human glucocorticoid receptor (hGR) binds steroid molecules that have diffused into the cell and the ligand-receptor complex translocates to the nucleus where transcriptional activation occurs.

A high-throughput screen of hGR translocation has distinct advantages over in vitro ligand-receptor binding assays because other parameters can be screened in parallel such as the function of other receptors, targets, or other cellular processes. Indicator cells, such as HeLa cells, are transiently transfected with a plasmid encoding GFP-hGR chimeric protein and the translocation of GFP-hGR into the nucleus is visualized.

EHR is used to introduce mutations into hGR to block signaling in normal and cancer cells and cells with aberrant ligand-receptor translocation are screened. The hGR gene is biovalidated by screening for drugs that enhance or revert the altered phenotype.

What we claim is:

1. A method of isolating a target nucleic acid comprising:
a) providing an enhanced homologous recombination (EHR) composition comprising: and
i) a recombinase;
ii) a first and a second targeting polynucleotide substantially complementary to each other, wherein said first targeting polynucleotide comprises a portion substantially complementary to a fragment of said targeting nucleic acid; and
iii) a separation moiety;
b) contacting said EHR composition with a library of target nucleic acid under conditions favoring hybridization wherein said first and/or said second targeting polynucleotides hybridize to at least one target nucleic acid of said library;
wherein said providing and/or contacting steps are performed using a robotic system comprising at least one module selected from the group consisting of:
1) a targeting polynucleotide synthesis module;
2) a target capture module;
3) a transformation and amplification module;
4) a clone verification module;
5) a DNA purification module;
6) a restriction analysis module;
7) a DNA sequencing module; and
8) a computer database module.

2. The method according to claim 1 wherein said target nucleic acid is a portion of a target gene.

3. The method according to claim 1 wherein said target nucleic acid is a regulatory sequence.

4. The method according to claim 1, wherein said target nucleic acid comprises a single-nucleotide polymorphism.

5. The method of claim 1, wherein said library of target nucleic acids comprises all or part of a cDNA library.

6. The method of claim 1, wherein said library of target nucleic acids comprises all or part of a genomic library.

7. The method of claim 5 or 6, wherein said cDNA library or said genomic library is from a single organism.

8. The method according to claim 1 further comprising:
d) making a library of nucleic acid variants of said target nucleic acid;
e) introducing said library of nucleic acid variants into cells to make a cellular library; and
f) performing phenotypic screening on said cellular library.

9. The method according to claim 8, wherein at least one of steps (d), (e), or (f) is performed using a robotic system wherein said robotic system comprises at least one of said modules selected from the group consisting of modules (1), (2), (3), (4), (5), (6), (7), and (8).

10. The method according to claim 1 further comprising:
d) making a plurality of cells comprising a mutant of said target nucleic acid;
e) adding a library of candidate agents to said plurality of cells;
f) determining the effect of said candidate agents on said cells.

11. The method according to claim 10, wherein at least one of steps (d), (e), or (f) is performed using a robotic system wherein said robotic system comprises at least one of said modules selected from the group consisting of modules (1), (2), (3), (4), (5), (6), (7), and (8).

12. The method according to claim 10, wherein said mutant of said target nucleic acid is a gene sequence knock-out, a gene sequence knock-in, a modification of nucleic acid regulatory sequence, or a modification of an intronic sequence.

13. The method according to claim 10, wherein said mutant of said target nucleic acid comprises an insertion, substitution, or deletion of one or more nucleotides to said target nucleic acid or combinations thereof.

14. A robotic system comprising:
(1) a computer workstation comprising a microprocessor programmed to manipulate a device selected from the group consisting of a thermocycler, a multichannel pipettor, a sample handler, a plate handler, a gel loading system, an automated transformation system, a gene sequencer, a colony picker, a bead picker, a cell sorter, an incubator, a light microscope, a fluorescence microscope, a spectrofluorometer, a spectrophotometer, a luminometer, a CCD camera and combinations thereof; and
(2) at least one module selected from the group consisting of:
a) a targeting polynucleotide synthesis module;
b) a target capture module;
c) a transformation and amplification module;
d) a clone verification module;
e) a DNA purification module;
f) a restriction analysis module;
g) a DNA sequencing module; and
h) a computer database module.

15. The robotic system of claim 14 specifically adapted for producing a plurality of enhanced homologous recombination compositions.

16. The robotic system of claim 15 specifically adapted for contacting compositions with a cellular library under conditions wherein said compositions hybridize to one or more target nucleic acid members of said library.

17. The robotic system of claim 16 further comprising means for isolating the target nucleic acids.

18. The robotic system of claim 16 further comprising means for producing a library of mutant target nucleic acid(s).

19. The robotic system of claim 16 further comprising means for nucleotide sequencing the target nucleic acid(s).

20. The robotic system of claim 16 further comprising means for determining the genotype of the target nucleic acid(s).

21. A method of high throughput integrated genomics comprising:
a) providing a plurality of enhanced homologous recombination (EHR) compositions, wherein each composition comprises:
i) a recombinase;
ii) a first and a second targeting polynucleotide, wherein said first targeting polynucleotide comprises a portion substantially complementary to a fragment of a target nucleic acid and is substantially complementary to said second targeting polynucleotide; and
iii) a separation moiety;
b) contacting said EHR compositions with one or more nucleic acid sample(s) under-conditions wherein said targeting polynucleotides hybridize to one or more target nucleic acid member(s) of one or more nucleic acid sample(s); and
c) isolating said target nucleic acid(s) wherein said providing, contacting and/or isolating are performed using a robotic system comprising at least one module selected from the group consisting of:
1) a targeting polynucleotide synthesis module;
2) a target capture module;
3) a transformation and amplification module;
4) a clone verification module;
5) a DNA purification module;
6) a restriction analysis module;
7) a DNA sequencing module; and
8) a computer database module.

22. The method according to claim 21, wherein said target nucleic acid is a portion of said target gene.

23. The method according to claim 21, wherein said target nucleic acid is a regulatory sequence.

24. The method according to claim 21 further comprising:
d) making a library of nucleic acid variants of said target nucleic acid;
e) introducing said library of nucleic acid variants into a cellular library; and
f) performing phenotypic screening on said cellular library.

25. The method according to claim 24 wherein at least one of said making, introducing and performing steps are performed using a robotic system wherein said robotic system is comprised of at least one of said modules selected from the group consisting of modules (1), (2), (3), (4), (5), (6), (7), and (8).

26. The method according to claim 21 further comprising:
d) making a plurality of cells comprising a mutant target nucleic acid;
e) adding a library of candidate agents to said plurality; and
f) determining the effect of said candidate agents on said cells.

27. The method according to claim 26, wherein said mutant target nucleic acid comprises an insertion, substitution, deletion or combinations thereof.

28. A method of isolating a target genomic DNA comprising:
a) providing an enhanced homologous recombination (EHR) composition comprising: and
i) a recombinase;
ii) a first and a second targeting polynucleotide substantially complementary to each other, wherein said first targeting polynucleotide comprises a portion substantially complementary to a portion of said target genomic DNA; and
iii) a separation moiety;
b) contacting said EHR composition with genomic DNA a library under conditions favoring hybridization wherein said first and/or said second targeting polynucleotides hybridize to said target genomic DNA;
wherein said providing and/or contacting steps are performed using a robotic system comprising at least one module selected from the group consisting of:
1) a targeting polynucleotide synthesis module;
2) a target capture module;
3) a transformation and amplification module;
4) a clone verification module;
5) a DNA purification module;
6) a restriction analysis module;
7) a DNA sequencing module; and
8) a computer database module.

29. The method of claim 28 wherein said target genomic DNA is separated from said genomic DNA.

30. The method of claim 28 wherein said target genomic DNA comprises a fragment of a genome.

31. The method of claim 28 wherein said target genomic DNA comprises a chromosome.

32. The method of claim 31, wherein said chromosome is mammalian.
